SEQUENCE LISTING (2 sequences)

SEQ ID NO: 1 - RNA, 23 nt, Homo sapiens
accccuaucacgauuagcauuaa

SEQ ID NO: 2 - RNA, 22 nt, Artificial Sequence
  Description of Artificial Sequence Synthetic oligonucleotide
uucacaaugcguuaucggaugu

(19) United States
(12) Patent Application Publication
(10) Pub. No.: US 2013/0236453 A1
(43) Pub. Date: Sep. 12, 2013

(54) Methods and Compositions for Modulating Acute Graft-versus-Host Disease using miR-155 Specific Inhibitors

(71) Applicant: THE OHIO STATE UNIVERSITY, Columbus, OH (US)

(72) Inventors: Carlo M. Croce, Columbus, OH (US); Ramiro Garzon, Columbus, OH (US)

(73) Assignee: THE OHIO STATE UNIVERSITY, Columbus, OH (US)

(21) Appl. No.: 13/795,879

(22) Filed: Mar. 12, 2013

Related U.S. Application Data
(60) Provisional application No. 61/609,752, filed on Mar. 12, 2012.

(51) Int. Cl. A61K 31/7088

(52) U.S. Cl. CPC A61K 31/7088
USPC 424/133.1; 514/44 A; 536/24.5; 424/278.1; 424/158.1; 424/173.1; 424/93.7; 424/549; 424/577; 424/571; 424/569; 424/574; 424/548; 424/558; 424/557; 424/553; 424/551; 424/556; 424/579; 424/580; 424/93.71

(57) ABSTRACT

Methods and compositions for treating or preventing acute graft-versus-host disease (aGVHD) in a subject using miR-155 specific inhibitors are described.

### CROSS-REFERENCE TO RELATED APPLICATIONS

This application claims the benefit of U.S. Provisional Patent Application Ser. No. 61/609,752 filed Mar. 12, 2012, the entire disclosure of which is expressly incorporated herein by reference for all purposes.

### STATEMENT REGARDING FEDERALLY SPONSORED RESEARCH

This invention was made with government support under Grant Nos. R01-172699, AI34495, R01-HL56067, and P50-CA140158 awarded by the National Institutes of Health. The U.S. government has certain rights in the invention.

### SEQUENCE LISTING

The instant application contains a Sequence Listing which has been submitted via EFS-web and is hereby incorporated by reference in its entirety. The ASCII copy, created on Feb. 28, 2013, is named 604_{—}53837_SEQ_LIST_{—}2012-194.txt, and is 726 bytes in size.

### TECHNICAL FIELD

This invention relates generally to the field of molecular biology. More particularly, it concerns approaches to enhance the efficacy and safety of allogeneic grafts. The disclosed miR-155 specific inhibitors, and methods of use thereof, do not cause general immunosuppression in a subject, and induce a significant reduction of acute graft-versus-host disease (aGVHD) in subjects.

### BACKGROUND

Acute GVHD (aGVHD) when allogeneic donor T cells destroy human leukocyte antigen (HLA) mismatched host tissues by secreting inflammatory cytokines (interleukin-1 (IL-1), tumor necrosis factor-α (TNF-α), and interferon-γ (IFN-γ)) and/or inducing direct cytotoxic cellular response.

aGVHD develops in 30-75% of recipients of allogeneic hematopoietic stem cell transplant (alloHSCT) and is associated with significant morbidity and mortality. Thus, aGVHD represents a major barrier towards the wider and safer application of this potentially curative approach to hematologic malignancies.

Current therapeutic interventions rely highly on immunosuppressive treatment. Immunosuppression may increase risks of infection and cancer relapse. It may also compromise the benefit of the graft. For instance, in leukemia, allogeneic lymphocytes produce a beneficial graft-versus-leukemia (GVL) effect, but the beneficial effect is limited by graft-versus-host disease (GVHD); efforts to prevent GVHD have resulted in reduced GVL effect.

In spite of considerable research into therapies to treat aGVHD, it remains difficult to treat effectively, and the mortality observed in patients indicates that improvements are needed in the diagnosis, treatment and prevention of this disease.

### SUMMARY

In a first broad aspect, there is provided herein a method of reducing an immune response against mis-matched transplanted organs/tissue/cells (e.g., bone marrow, heart, kidney, liver, lung, pancreas, peripheral stem cells) by administering a miR specific inhibitor to the recipient subject.

In another aspect, there is provided herein a method for the treatment of bone marrow or peripheral stem cell transplant donors with a miR gene product to attenuate the development of graft-versus-host-disease in the recipients.

In one aspect there is provided herein, a method for treating or preventing acute graft-versus-host disease (aGVHD) in a subject, comprising: administering to the subject a miR-155 specific inhibitor in an amount effective to decrease expression of miR-155 in the subject.

In another aspect there is provided herein, a method for treating acute graft-versus-host disease (aGVHD) in a recipient of an organ or tissue transplant, comprising: administering to the transplant recipient a miR-155 specific inhibitor in a pharmaceutically effective amount after the transplantation.

In another aspect there is provided herein, a method for decreasing a T-cell activity in an individual suffering from acute graft-versus-host disease comprising: administering to the individual an effective amount of a miR-155 specific inhibitor, and a pharmaceutically acceptable carrier; wherein the miR-155 specific inhibitor decreases a T-cell activity in vivo.

In another aspect there is provided herein, a method of inhibiting miR-155 expression in a subject having, or at risk of having acute graft-versus-host disease (aGVHD), comprising: introducing an effective amount of at least one miR-155-specific inhibitor to the subject.

In another aspect there is provided herein, a method of treating aGVHD in a subject, comprising: administering to the subject a therapeutically effective amount of a miR-155 inhibitor to treat or ameliorate one or more symptoms of aGVHD.

In another aspect there is provided herein, a method of treating tissue or organ transplant rejection in a recipient comprising the step of parenterally administering to the recipient an effective amount of a miR-155 specific inhibitor composition to attenuate the tissue or organ transplant rejection.

In another aspect there is provided herein, a method of modulating the immune response against an organ or tissue transplant in a recipient comprising the step of parenterally administering to the recipient an effective amount of a miR-155 specific inhibitor composition to modulate the response against the organ or tissue transplant.

In another aspect there is provided herein, a method of treating graft-versus-host-disease in a recipient comprising the step of parenterally administering to the recipient an effective amount of a miR-155 specific inhibitor composition to attenuate the graft-versus-host-disease in the recipient.

In certain embodiments, the miR-155 specific inhibitor partially suppresses TNF-α expression, thereby treating or preventing aGVHD.

In certain embodiments, the at least one miR-155-specific inhibitor is selected from the group consisting of pre-miR-155, pri-miR-155, mature miR-155, and seed miR-155.

In certain embodiments, the miR-specific inhibitor is selected from the group consisting of anti-miRs and target mimics.

In certain embodiments, the miR-155 specific inhibitor is an anti-miR-155 antisense oligonucleotide.

In certain embodiments, the antisense oligonucleotide is an antagomir.

In certain embodiments, the miR-155 inhibitor is administered to a subject at risk for aGVHD

In certain embodiments, the miR-155 inhibitor decreases levels of TNF-α in cells of the subject.

In certain embodiments, the miR-155 inhibitor is an antisense oligonucleotide that is complementary to miR-155.

In certain embodiments, the antisense oligonucleotide is complementary to at least 10 contiguous nucleotides in miR-155.

In certain embodiments, the antisense oligonucleotide comprises between 7 and 25 nucleotides.

In certain embodiments, the antisense oligonucleotide comprises between 7 and 21 nucleotides.

In certain embodiments, the miR-specific inhibitor comprises a nucleotide sequence of least 6 consecutive nucleotides that are complementary to the positions 2-8 of the seed region of LNA-miR-155 specific inhibitor, and wherein the miR-specific inhibitor reduces levels of TNF-α.

In certain embodiments, the miR-specific inhibitor is chemically modified on at least one nucleotide.

In certain embodiments, the chemical modification comprises a locked nucleic acid (LNA).

In certain embodiments, one or more of the nucleotide units of the antisense oligonucleotide are locked nucleic acid (LNA) units or 2′ substituted nucleotide analogues.

In certain embodiments, one or more of the internucleoside linkages between the nucleotide units of the antisense oligonucleotide are phosphorothioate internucleoside linkages.

In certain embodiments, the chemical modification comprises 2′-O-methyl.

In certain embodiments, the miR-specific inhibitor comprises a polynucleic acid molecule that is essentially complementary to miR-155.

In certain embodiments, the miR-specific inhibitor comprises a polynucleic acid molecule that is 100% complementary to miR-155.

In certain embodiments, the miR-155 specific inhibitor is complementary to a sequence at least 80% identical to human mature miR-155.

In certain embodiments, the miR-155 specific inhibitor is complementary to a sequence at least 80% identical to pre-miR-155.

In certain embodiments, the miR-155 specific inhibitor is perfectly complementary to a human miR-155 seed sequence.

In certain embodiments, the subject transplant recipient has aGVHD.

In certain embodiments, the subject transplant recipient is at risk of developing aGVHD.

In certain embodiments, the transplant recipient has received hematopoietic stem cell transplant or bone marrow transplant.

In certain embodiments, the transplant recipient has received hematopoietic stem cell transplant or bone marrow transplant and manifests grade 2 or greater a GVHD.

In certain embodiments, the miR-155 specific inhibitor is administered orally to the subject transplant recipient.

In certain embodiments, the miR-155 specific inhibitor is administered parenterally to the subject transplant recipient.

In certain embodiments, the miR-155 specific inhibitor is administered to the subject transplant recipient via intravenous infusion or injection.

In certain embodiments, the subject is a mammal.

In certain embodiments, the subject is a human.

In certain embodiments, the miR-specific inhibitor comprises LNA-miR-155 specific inhibitor.

In another aspect there is provided herein, a pharmaceutical formulation comprising a miR-155 specific inhibitor.

In certain embodiments, the pharmaceutical composition is formulated for controlled or sustained release.

In another aspect there is provided herein, a miR-155 specific inhibitor for use in treating aGVHD in a subject.

In certain embodiments, the miR-155 specific inhibitor composition is dispersed in a pharmaceutically acceptable carrier.

In certain embodiments, the miR-155 specific inhibitor is mammalian miR-155 specific inhibitor.

In certain embodiments, the miR-155 specific inhibitor is human miR-155 specific inhibitor.

In certain embodiments, the miR-155 specific inhibitor is recombinant miR-155 specific inhibitor.

In certain embodiments, the method further comprises administering the miR-155 specific inhibitor in a delayed release formulation.

In certain embodiments, the amount of the miR-155 specific inhibitor composition that is administered is about 1 mg to about 20 g per day.

In certain embodiments, the amount of the miR-155 specific inhibitor composition that is administered is about 0.1 g to about 5 g per day.

In another aspect there is provided herein, a method of transplanting an allogeneic graft into a human subject in need thereof, comprising:

administering to the subject an effective amount of one or more of:

In one aspect there is provided herein, a method for treating or preventing acute graft-versus-host disease (aGVHD) in a subject, comprising: administering to the subject a miR-155 specific inhibitor in an amount effective to decrease expression of miR-155 in the subject.

In another aspect there is provided herein, a method for treating acute graft-versus-host disease (aGVHD) in a recipient of an organ or tissue transplant, comprising: administering to the transplant recipient a miR-155 specific inhibitor in a pharmaceutically effective amount after the transplantation.

In another aspect there is provided herein, a method for decreasing a T-cell activity in an individual suffering from acute graft-versus-host disease comprising: administering to the individual an effective amount of a miR-155 specific inhibitor, and a pharmaceutically acceptable carrier; wherein the miR-155 specific inhibitor decreases a T-cell activity in vivo.

In another aspect there is provided herein, a method of inhibiting miR-155 expression in a subject having, or at risk of having acute graft-versus-host disease (aGVHD), comprising: introducing an effective amount of at least one miR-155-specific inhibitor to the subject.

In another aspect there is provided herein, a method of treating aGVHD in a subject, comprising: administering to the subject a therapeutically effective amount of a miR-155 inhibitor to treat or ameliorate one or more symptoms of aGVHD.

In another aspect there is provided herein, a method of treating tissue or organ transplant rejection in a recipient comprising the step of parenterally administering to the recipient an effective amount of a miR-155 specific inhibitor composition to attenuate the tissue or organ transplant rejection.

In another aspect there is provided herein, a method of modulating the immune response against an organ or tissue transplant in a recipient comprising the step of parenterally administering to the recipient an effective amount of a miR-155 specific inhibitor composition to modulate the response against the organ or tissue transplant.

In another aspect there is provided herein, a method of treating graft-versus-host-disease in a recipient comprising the step of parenterally administering to the recipient an effective amount of a miR-155 specific inhibitor composition to attenuate the graft-versus-host-disease in the recipient.

In certain embodiments, the miR-155 specific inhibitor partially suppresses TNF-α expression, thereby treating or preventing aGVHD.

In certain embodiments, the at least one miR-155-specific inhibitor is selected from the group consisting of pre-miR-155, pri-miR-155, mature miR-155, and seed miR-155.

In certain embodiments, the miR-specific inhibitor is selected from the group consisting of anti-miRs and target mimics.

In certain embodiments, the miR-155 specific inhibitor is an anti-miR-155 antisense oligonucleotide.

In certain embodiments, the antisense oligonucleotide is an antagomir.

In certain embodiments, the miR-155 inhibitor is administered to a subject at risk for aGVHD

In certain embodiments, the miR-155 inhibitor decreases levels of TNF-α in cells of the subject.

In certain embodiments, the miR-155 inhibitor is an antisense oligonucleotide that is complementary to miR-155.

In certain embodiments, the antisense oligonucleotide is complementary to at least 10 contiguous nucleotides in miR-155.

In certain embodiments, the antisense oligonucleotide comprises between 7 and 25 nucleotides.

In certain embodiments, the antisense oligonucleotide comprises between 7 and 21 nucleotides.

In certain embodiments, the miR-specific inhibitor comprises a nucleotide sequence of least 6 consecutive nucleotides that are complementary to the positions 2-8 of the seed region of LNA-miR-155 specific inhibitor, and wherein the miR-specific inhibitor reduces levels of TNF-α.

In certain embodiments, the miR-specific inhibitor is chemically modified on at least one nucleotide.

In certain embodiments, the chemical modification comprises a locked nucleic acid (LNA).

In certain embodiments, one or more of the nucleotide units of the antisense oligonucleotide are locked nucleic acid (LNA) units or 2′ substituted nucleotide analogues.

In certain embodiments, one or more of the internucleoside linkages between the nucleotide units of the antisense oligonucleotide are phosphorothioate internucleoside linkages.

In certain embodiments, the chemical modification comprises 2′-O-methyl.

In certain embodiments, the miR-specific inhibitor comprises a polynucleic acid molecule that is essentially complementary to miR-155.

In certain embodiments, the miR-specific inhibitor comprises a polynucleic acid molecule that is 100% complementary to miR-155.

In certain embodiments, the miR-155 specific inhibitor is complementary to a sequence at least 80% identical to human mature miR-155.

In certain embodiments, the miR-155 specific inhibitor is complementary to a sequence at least 80% identical to pre-miR-155.

In certain embodiments, the miR-155 specific inhibitor is perfectly complementary to a human miR-155 seed sequence.

In certain embodiments, the subject transplant recipient has aGVHD.

In certain embodiments, the subject transplant recipient is at risk of developing aGVHD.

In certain embodiments, the transplant recipient has received hematopoietic stem cell transplant or bone marrow transplant.

In certain embodiments, the transplant recipient has received hematopoietic stem cell transplant or bone marrow transplant and manifests grade 2 or greater a GVHD.

In certain embodiments, the miR-155 specific inhibitor is administered orally to the subject transplant recipient.

In certain embodiments, the miR-155 specific inhibitor is administered parenterally to the subject transplant recipient.

In certain embodiments, the miR-155 specific inhibitor is administered to the subject transplant recipient via intravenous infusion or injection.

In certain embodiments, the subject is a mammal.

In certain embodiments, the subject is a human.

In certain embodiments, the miR-specific inhibitor comprises LNA-miR-155 specific inhibitor.

In another aspect there is provided herein, a pharmaceutical formulation comprising a miR-155 specific inhibitor.

In certain embodiments, the pharmaceutical composition is formulated for controlled or sustained release.

In another aspect there is provided herein, a miR-155 specific inhibitor for use in treating aGVHD in a subject.

In certain embodiments, the miR-155 specific inhibitor composition is dispersed in a pharmaceutically acceptable carrier.

In certain embodiments, the miR-155 specific inhibitor is mammalian miR-155 specific inhibitor.

In certain embodiments, the miR-155 specific inhibitor is human miR-155 specific inhibitor.

In certain embodiments, the miR-155 specific inhibitor is recombinant miR-155 specific inhibitor.

In certain embodiments, the method further comprises administering the miR-155 specific inhibitor in a delayed release formulation.

In certain embodiments, the amount of the miR-155 specific inhibitor composition that is administered is about 1 mg to about 20 g per day.

In certain embodiments, the amount of the miR-155 specific inhibitor composition that is administered is about 0.1 g to about 5 g per day.

In another aspect there is provided herein, a method of transplanting an allogeneic graft into a human subject in need thereof, comprising: administering to the subject an effective amount of one or more of: i) at least one miR-155 specific inhibitor; ii) active allo-reactive donor human cells which do not express sufficient miR-155 to upregulate T cell expression in the human subject; and, iii) active auto-reactive human cells which do not express sufficient miR-155 to upregulate T cell expression in the human subject; and, transplanting the allogeneic graft into the subject.

In another aspect there is provided herein, a method of preventing or reducing acute graft-versus-host disease (aGVHD) in a human subject undergoing allogeneic graft, comprising: administering to the subject an effective amount of one or more of: i) at least one miR-155 specific inhibitor; ii) active allo-reactive donor human cells which do not express sufficient miR-155 to upregulate T cell expression in the human subject; and, iii) active auto-reactive human cells which do not express sufficient miR-155 to upregulate T cell expression in the human subject; and, transplanting the allogeneic graft into the subject, wherein the administration of one or more of i), ii) and iii) prevents or reduces aGVHD in the subject.

In another aspect there is provided herein, a method of treating or preventing an infection in a subject undergoing allogeneic graft, comprising: administering to the subject an effective amount of one or more of: i) at least one miR-155 specific inhibitor; ii) active allo-reactive donor human cells which do not express sufficient miR-155 to upregulate T cell expression in the human subject; and, iii) active auto-reactive human cells which do not express sufficient miR-155 to upregulate T cell expression in the human subject; and, transplanting into the subject the allogeneic graft, wherein the administration of one or more of i), ii) and iii) prevents or reduces aGVHD in the subject.

In another aspect there is provided herein, a method of increasing engraftment or for reducing graft rejection in a human subject undergoing allogeneic graft, comprising: administering to the subject an effective amount of one or more of: i) at least one miR-155 specific inhibitor; ii) active allo-reactive donor human cells which do not express sufficient miR-155 to upregulate T cell expression in the human subject; and, iii) active auto-reactive human cells which do not express sufficient miR-155 to upregulate T cell expression in the human subject; and, transplanting into the human subject the allogeneic graft, wherein the administration of one or more of i) and ii) prevents or reduces aGVHD in the subject.

In certain embodiments, one or more of: i) the miR-155 specific inhibitor; ii) active allo-reactive donor human cells; and, iii) active auto-reactive human cells; and, the allogeneic graft are administered into the human subject simultaneously.

In certain embodiments, one or more of: i) the miR-155 specific inhibitor; ii) active allo-reactive donor human cells; and, iii) active auto-reactive human cells are administered prior to the allogeneic graft.

In certain embodiments, the active allo-reactive cells are prepared by: providing cells from an allo-reactive human donor; removing a substantial amount of miR-155 from the cells; and, collecting the miR-155 reduced cells.

In certain embodiments, the graft is bone, bone marrow, cornea, heart valve, skin, tendons, stem cells or veins.

In certain embodiments, the graft is eye, kidney, heart, lung, liver, intestine, pancreas, spleen or thymus.

In certain embodiments, the method further comprises treating ex vivo the donor organ or tissue with the miR-155 specific inhibitor prior to transplantation in the subject: i) at least one miR-155 specific inhibitor; ii) active allo-reactive donor human cells which do not express sufficient miR-155 to upregulate T cell expression in the human subject; and, iii) active auto-reactive human cells which do not express sufficient miR-155 to upregulate T cell expression in the human subject; and, transplanting the allogeneic graft into the subject.

In another aspect there is provided herein, a method of preventing or reducing acute graft-versus-host disease (aGVHD) in a human subject undergoing allogeneic graft, comprising:

administering to the subject an effective amount of one or more of: i) at least one miR-155 specific inhibitor; ii) active allo-reactive donor human cells which do not express sufficient miR-155 to upregulate T cell expression in the human subject; and, iii) active auto-reactive human cells which do not express sufficient miR-155 to upregulate T cell expression in the human subject; and, transplanting the allogeneic graft into the subject, wherein the administration of one or more of i), ii) and iii) prevents or reduces aGVHD in the subject.

In another aspect there is provided herein, a method of treating or preventing an infection in a subject undergoing allogeneic graft, comprising: administering to the subject an effective amount of one or more of: i) at least one miR-155 specific inhibitor; ii) active allo-reactive donor human cells which do not express sufficient miR-155 to upregulate T cell expression in the human subject; and, iii) active auto-reactive human cells which do not express sufficient miR-155 to upregulate T cell expression in the human subject; and, transplanting into the subject the allogeneic graft, wherein the administration of one or more of i), ii) and iii) prevents or reduces aGVHD in the subject.

In another aspect there is provided herein, a method of increasing engraftment or for reducing graft rejection in a human subject undergoing allogeneic graft, comprising: administering to the subject an effective amount of one or more of: i) at least one miR-155 specific inhibitor; ii) active allo-reactive donor human cells which do not express sufficient miR-155 to upregulate T cell expression in the human subject; and, iii) active auto-reactive human cells which do not express sufficient miR-155 to upregulate T cell expression in the human subject; and, transplanting into the human subject the allogeneic graft, wherein the administration of one or more of i) and ii) prevents or reduces aGVHD in the subject.

In certain embodiments, one or more of: i) the miR-155 specific inhibitor; ii) active allo-reactive donor human cells; and, iii) active auto-reactive human cells; and, the allogeneic graft are administered into the human subject simultaneously.

In certain embodiments, one or more of: i) the miR-155 specific inhibitor; ii) active allo-reactive donor human cells; and, iii) active auto-reactive human cells are administered prior to the allogeneic graft.

In certain embodiments, the active allo-reactive cells are prepared by: providing cells from an allo-reactive human donor; removing a substantial amount of miR-155 from the cells; and, collecting the miR-155 reduced cells.

In certain embodiments, the graft is bone, bone marrow, cornea, heart valve, skin, tendons, stem cells or veins.

In certain embodiments, the graft is eye, kidney, heart, lung, liver, intestine, pancreas, spleen or thymus.

In certain embodiments, the method further comprises treating ex vivo the donor organ or tissue with the miR-155 specific inhibitor prior to transplantation in the subject.

In another aspect there is provided herein, a method comprising treating a subject undergoing cancer treatment, by: providing immunocompetent donor cells; inducing graft versus tumor (GvT) response in the subject by transfusing the donor cells to the subject; and mitigating aGVHD by administering a miR-155 specific inhibitor.

In certain embodiments, the a cancer being treated is selected from the group consisting of: leukemia, acute myeloid leukemia (AML), chronic lymphocytic leukemia (CLL), chronic myelogenous leukemia (CML), lymphoma, myeloma, and neuroblastoma. In certain embodiments the subject is immunocompromised at the time of transfusion; and, the donor cells are T cell replete. In certain embodiments, the donor cells comprise, or are derived from, at least one of: T cells, hematopoietic stem cells, bone marrow, spleen, peripheral blood, umbilical cord blood, amniotic fluid, and dental pulp. In certain embodiments, the miR-155 specific inhibitor comprises an 8-mer seed-targeting LNA-antimiR-155 with the sequence: 5′-TAGCATTA-3′.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims

### BRIEF DESCRIPTION OF THE FIGURES

The patent or application file may contain one or more drawings executed in color and/or one or more photographs. Copies of this patent or patent application publication with color drawing(s) and/or photograph(s) will be provided by the Patent Office upon request and payment of the necessary fee.

FIGS. 1A-1D. MicroRNA-155 Expression is Up-Regulated in Effector T Cells of Mice Recipients with Acute GVHD.

FIG. 1A. Schematic presentation depicting aGVHD murine model used.

FIG. 1B. Histopathologic evaluation of a representative liver sample collected from a mouse with clinical score of GVHD of ≧7. This section was stained with H&E. Magnification is 40× and 400×.

FIG. 1C. Average miR-155 expression in CD4⁺ CD62L⁻ T cells isolated from the spleen of three recipient mice with aGVHD (BM+spleen) or controls (BM alone). The results shown represent the average of three independent mouse samples performed each in triplicate. Values are expressed as relative miRNA expression after 2ΔCt calculations and normalization with sno135. Bars represent standard deviation.

FIG. 1D. Hierarchical clustering analysis of miRNA expression obtained using Nanostring from CD4⁺ CD62L⁻ T cells isolated from the spleen of recipient mice with aGVHD (BM+spleen) or controls (BM alone). Color areas indicate relative expression of each miRNA among the two types of samples (red high, blue low expression).

FIG. 1E. Histopathologic and molecular evaluation of a representative liver sample collected from a mouse with clinical score of GVHD of ≧7 or a control mouse (BM alone). At least 3 mice in each group were evaluated for staining with LNA anti-miR-155 and scramble control. These sections were stained with LNA anti-miR-155 probes or scrambled controls. Dark staining means positivity. Magnification is 400×. Note the strong staining of the mononuclear cells in the periportal lymphoid aggregate in the BM+spleen mouse that is lost in the same cells in the serial section with the scrambled probe.

FIG. 2A-2H. Recipients from miR-155 Deficient Splenocytes do not Develop Severe Acute GVHD and have Increased Survival.

FIG. 2A. Schematic presentation depicting the different donor cells used in lethally irradiated F1 mice.

FIG. 2B. Clinical scores from the different recipient mice groups post transplant.

FIG. 2C. Survival rate of lethally irradiated mice receiving BM alone, BM+miR-155^{-/-} spleen, BM+miR-155^{+/+} spleen or XRT alone. Data shown here represents two independent experiments.

FIG. 2D. Histopathological GVHD scores of recipient tissues. A second cohort of mice was used for histopathological analysis. After 15 days post transplantation (except for XRT group), mice from the indicated groups were sacrificed and sections of the large bowel and liver were stained with H&E. Data are pooled from 2 independent experiments, representing about 6 or 7 animals per group, the XRT group that had 4 mice. There was no significant difference in the liver GVHD scores between the BM alone and BM+miR-155^{-/-} spleen recipients (P=0.07). The GVHD scores in the gut were higher in the BM+miR-155^{-/-} spleen recipients versus the BM alone (P=0.04).

FIG. 2E. Histopathologic evaluation of representative liver and large bowel samples collected from the different recipient mice groups at day 15 post transplant. The arrows in the large bowel slide from the B6 BM+miR-155^{+/+} spleen F1 recipients showed lymphoplasmacytic infiltration in the lamina propia with frequent apoptosis. The arrows in the liver slide from the same F1 recipients showed abundant lymphocytes infiltrating around the portal vein. No significant pathology is shown in BM alone and miR-155^{-/-} groups.

FIG. 2F. Clinical GVHD scores post transplant from lethally irradiated B10.BR recipients transplanted with BM alone, BM+miR-155^{-/-} or BM+miR-155^{+/+} spleen.

FIG. 2G. Survival rate of the different recipient B 10.BR mice groups.

FIG. 2H. TNF-α levels measured in the serum of lethally irradiated F1 recipients of BM alone, BM+miR-155^{-/-} spleen and BM+miR-155^{+/+} spleen, 15 days after transplant. Samples were assayed in triplicate and results are shown as the means from four biological samples within a group.

FIGS. 3A-3G. Recipient Mice from Donor Splenocytes Over-Expressing miR-155 in T Cells Exhibit Rapidly Evolving Acute GVHD and Short Survival.

FIG. 3A. Schematic presentation showing the construct used to generate the LCK-miR-155 mice.

FIG. 3B. miR-155 expression (Fold Change) in the thymus and spleen from age and sex matched B6 LCK-miR-155 transgenic mice and B6 miR-155^{-/-} mice. Bars represent SD.

FIG. 3C. Schematic presentation depicting the different recipients F1 mice groups.

FIG. 3D. Clinical GVHD scores post transplant from lethally irradiated F1 recipients transplanted with BM alone (n=8), BM+LCK-miR-155 (n=8) or BM+miR-155^{+/+} (n=8) splenocytes. The P values were obtained by using t-test.

FIG. 3E. Survival rate of the different recipient F1 mice groups. Comparisons between BM+miR-155^{-/-} spleen and BM+miR-155^{+/+} spleen survival curves was performed using log-rank test.

FIG. 3F. Histopathological GVHD scores of recipient tissues. A second cohort of mice was used for histopathological analysis. After 14 days post transplantation mice from the indicated groups were sacrificed and sections of the large bowel and liver were stained with H&E. The P values were obtained by using t-test.

FIG. 3G. Histopathologic evaluation of representative liver samples obtained from recipients of BM alone, BM+miR-155^{+/+} spleen and BM+LCK-miR-155 donor cells. Slides were stained with H&E. The arrows show lymphocytic infiltration in the recipient livers characteristic of GVHD.

FIGS. 4A-4G. miR-155 Modulates Chemokine Receptor Expression and Migration of Allogeneic Donor T Cells.

On days 4 and 21 after BMT, splenocytes were harvested from F1 recipients and H2 Kd− CD4⁺/CD8⁺ donor cells were analyzed by flow cytometry for chemokine receptor expression. Chemokine receptor expression was quantified by measuring mean fluorescence intensity (MFI). P values <0.05 were considered significant. n=at least 5 mice per group at each time point.

FIG. 4A. Expression of CXCR4 on CD4⁺ and CD8⁺ WT and miR-155^{-/-} donor T cells on day 21 post transplant.

FIG. 4B. Expression of S1P1 on CD4⁺ WT and miR-155^{-/-} donor T cells on day 21 post transplant.

FIG. 4C. Expression of CCR5 on CD4⁺ and CD8⁺ WT and miR-155^{-/-} donor T cells on day 21 post transplant.

FIG. 4D. Average IL-12Rb1 mRNA expression in untouched T cells isolated from the spleen from recipient mice on day 21 post-transplant by RT-PCR. The results shown represent the average of nine independent mouse samples performed each in triplicate. Values are expressed as relative expression after 2ΔCt calculations and normalization with 18s RNA. Bars represent standard deviation.

FIG. 4E. Average STAT-4 mRNA expression in untouched T cells isolated from the spleen from recipient mice on day 21 post-transplant by RT-PCR.

FIG. 4F. Average IFN-γ mRNA expression in untouched T cells isolated from the spleen from recipient mice on day 21 post-transplant by RT-PCR.

FIG. 4G. Average CCR5 mRNA expression in untouched T cells isolated from the spleen from recipient mice on day 21 post-transplant by RT-PCR.

FIGS. 5A-5D. Treatment with LNA-antimiR-155 Decreases the Severity of aGVHD and Prolongs the Survival of MHC-Mismatched Recipient Mice.

FIG. 5A. Schematic presentation of the schedule of oligonucleotide injections post transplant. Briefly, lethally irradiated recipient F1 mice transplanted with BM (5×10⁶)+B6 WT spleen cells (20×10⁶) were treated with LNA-antimiR-155 (n=10) or control LNA oligos (n=10) starting at day 7 with a loading dose 25 mg/kg followed by 5 mg/Kg I.V. twice weekly up to day 30 after infusion of donor B6 splenocytes.

FIG. 5B. Clinical GVHD scores post transplant from lethally irradiated F1 recipients transplanted with B6 donor BM and splenocytes and treated with LNA-antimiR-155 or control oligonucleotide. The P values were obtained by using t-test.

FIG. 5C. Survival rate of the different recipient F1 mice groups. Comparisons between survival curves were performed using log-rank test.

FIG. 5D. miR-155 expression in spleen from LNA-antimiR-155 or control treated mice as measured by RT-PCR. Data represent the average of 3 mice. Experiments performed in triplicate. Bars represent standard error.

FIGS. 6A-6C. miR-155 Deficiency in Donor T Cells does not Abrogate GVL Effects.

FIG. 6A. Whole body bioluminescent signal intensity of recipient mice. Mice were imaged on day 10 and day 20.

FIG. 6B. Histopathological analysis of spleen and liver tissues of the different mice cohorts. Histopathological examination at lower magnification (100×) revealed extensive nodular leukemic infiltration in the liver in the BM alone recipients, while no infiltrates were seen in recipients who received splenocytes, either from WT or miR-155 deficient mice (Upper panels). In the middle upper panels the same liver sections are shown at higher magnification (200×). Note in the BM alone the many large anaplastic cells with pleomorphic, multilobular large nuclei and frequent mitosis figures. The last two lower panels showed spleen sections at lower (top) and higher (bottom) magnification. Note the complete replacement of the spleen by the large anaplastic cells in the BM alone group. The arrows point to normal spleen. There were no leukemic infiltrations in the recipient groups who received either WT or miR-155 deficient splenocytes.

FIG. 6C. Survival of lethally irradiated mice transplanted with WT or miR-155 deficient splenocytes+BM P815 leukemic cells. Survival comparisons were performed using the log-rank test.

FIGS. 7A-7J. miR-155 Expression is Up-Regulated in the Intestinal Tract from Patients with aGVHD.

Histopathological assessment of human small and large bowel samples from patients with aGVHD or from control patients who had a bowel biopsy but no pathology was observed (controls). In situ hybridization was performed using a digoxigenin-labeled LNA-modified probe complementary to miR-155 or a scrambled LNA control probe.

FIG. 7A. The colon tissue shows marked inflammation with a loss of glands and concomitant erosion. Note the many inflammatory cells in the lamina propria were positive for miR-155 (Dark staining). Magnification is 200×.

FIG. 7B. The miR-155 in situ hybridization signal is lost in the same cells in the serial section upon using the LNA scramble control probe.

FIG. 7C. A section of ileum shows a normal villous to the left side of the panel, and the loss of the villi in the rest of the section. Note the strong signal in the inflammatory cells in the area of the damaged villi with the miR-155 probe. Magnification is 200×.

FIG. 7D. Negative control for FIG. 7C, showing loss of the signal with the scrambled probe in the same cells in the serial section.

FIG. 7E and FIG. 7F. The area to the right of the section of ileum show in FIG. 7C is magnified (400×) in FIG. 7E (miR-155) and FIG. 7F (scrambled probe) to underscore the localization of the signal to mononuclear cells.

FIG. 7G. An earlier stage of GVHD with degenerated/regenerating glands with mononuclear infiltrates that are primarily located in the lamina propria adjacent to the damaged glands.

FIG. 7H. The miR-155 signal is rare in the damaged epithelial cells but is strong in the adjacent inflammatory cells as shown in higher magnification (1000×), where mononuclear infiltrate into the epithelia, typical of GVHD, is evident.

FIG. 7I and FIG. 7J. Normal colon mucosa negative for miR-155 staining. Magnification is 400×.

FIG. 8. miRNA-155 expression in CD8⁺ CD44 high T cells isolated from the spleen of recipient mice with aGVHD (BM+spleen) or controls (BM alone). CD8⁺ T cells from three mice with aGVHD (GVHD score ≧7) were magnetically separated from the spleen and stained with the indicated antibodies. The cells were sorted in CD8⁺ CD44high (upper panel) or CD8⁺ CD44low (lower panel) populations. Total RNA was isolated from the CD8⁺ CD44high (activated effected cells) and miR-155 expression was performed using Taqman miRNA assay. The results shown represent the average of three independent samples, performed each in triplicate. Values are expressed as relative miRNA expression after 2ΔCt calculations and normalization with sno135. Bars represent standard deviation.

FIG. 9. MicroRNA expression in wild type (miR-155^{+/+}) or miR-155 deficient (miR-155^{-/-}) CD4⁺ T cells at baseline and after T cell activation with CD3 and CD28. Untouched CD4⁺ T cells were isolated from the above recipients using magnetic separation and were cultured with CD3 and CD28 antibodies for 48 hours. Total RNA was isolated at baseline and after 48 hours of culture. miR-155 was measured by RT-PCR. The results shown represent the average of three independent samples, performed each in triplicate. Values are expressed as relative miRNA expression after 2ΔCt calculations and normalization with sno135. Bars represent standard deviation.

FIGS. 10A-10D. Recipients from miR-155 deficient splenocytes or T cells obtained from a second miR-155 deficient murine model do not develop severe acute GVHD and have increased survival. For this experiment we performed the B6 into F1 parent MHC model but using as a donor a second B6 miR-155 deficient mouse model (B6 miR-155-deficient^{m2/m2}).

FIG. 10A. Clinical scores from the different recipient mice groups post transplant according to the criteria published by Cooke et al. The P values were obtained by using t-test.

FIG. 10B. Survival rate of lethally irradiated mice receiving BM alone, BM+miR-155^{-/-} spleen, BM+miR-155^{+/+} spleen or XRT alone. Data shown here represents two independent experiments, using 3 mice each. Comparisons between BM+miR-155^{-/-} spleen and BM+miR-155^{+/+} spleen survival curves was performed using log-rank test. In order to eliminate the role of donor non-T cells in contributing to the phenotype seen in the aGVHD transplant recipients, the transplant experiment was repeated using 5×10⁶ T cell depleted BM cells and 2×10⁶ T cells isolated from the splenocytes of either miR-155^{+/+} or miR-155^{+/+} mice.

FIG. 10C. Clinical scores from the different recipient mice groups post transplant of donor T cells according to the criteria published by Cooke et al. The P values were obtained by using t-test.

FIG. 10D. Survival rate of lethally irradiated mice receiving BM alone, BM+miR-155^{-/-} T cells, BM+miR-155^{+/+} T cells or XRT alone.

FIGS. 11A-11B. Recipient mice from donor splenocytes over-expressing miR-155 in T cells exhibit increased serum cytokine levels.

FIG. 11A. miR-155 expression levels in the lymphocytes subpopulations and myeloid compartment in the bone marrow (BM) from LCK-miR-155 transgenic mice. BM was isolated from three LCK-miR-155 mice or age matched controls, and T, B and Myeloid cells were isolated using cell sorting. Total RNA was isolated from these cell populations and miR-155 expression was measured by RT-PCR as described in FIG. 9.

FIG. 11B. Cytokines levels (TNF-α, 11-2 and IFN-γ) were measured in the serum of lethally irradiated F1 recipients of BM alone, BM+miR-155^{+/+} spleen and BM+LCK-miR-155 spleen, 15 days after transplant by using ELISA assays. Samples were assayed in triplicate and results are shown as the means from four biological samples within a group. Bars represent standard deviation. P values were obtained using t-test.

FIGS. 12A-12C. miR-155 modulates chemokine receptor expression and migration of allogeneic donor T cells. On day 21 after BMT, splenocytes were harvested from F1 recipients and H2 Kd− CD4⁺/CD8⁺ donor cells were analyzed by flow cytometry for chemokine receptor expression. Chemokine receptor expression was quantified by measuring mean fluorescence intensity (MFI). 5 mice per group at each time point were analyzed.

FIG. 12A. Expression of CCR5 on CD4⁺ and CD8⁺ WT and miR-155^{-/-} donor T cells on day 21 post transplant.

FIG. 12B. Expression of CXCR4 on CD4⁺ and CD8⁺ WT and miR-155^{-/-} donor T cells on day 21 post transplant.

FIG. 12C. Expression of S1P1 on CD4⁺ WT and miR-155^{-/-} donor T cells on day 21 post transplant.

FIGS. 13A-13F. In vitro and in vivo proliferation and homeostatic assays.

FIG. 13A. Donor T cell activation (CD25 and CD69 expression) of B6 WT or miR155−/− CD4+ T cells isolated from splenocytes and cultured with BM derived F1 dendritic cells (DCs, allogeneic) or B6 DCs (syngeneic, negative control). CD69 and CD25 expression was measured after 4 days in culture as marker for activation. There was no difference in the expression of CD25 and CD69 between B6 WT or miR155−/− CD4+ T cells on allogeneic stimulation. One representative experiment of 4 is shown.

FIG. 13B. Lymphocyte proliferation assays. CFSE labeled CD4+ WT or miR155−/− T cells were cultured with F1 (allogeneic) or B6 (syngeneic, negative control) BM derived DCs. There was no significant difference in proliferation as shown by CFSE peaks 7 days after culture.

FIG. 13C. Supernatant cytokine measurements after 4 and 7 days of in vitro culture of B6 WT or miR155−/− CD4+ T cells cultured with B6 (syngeneic, negative control) or F1 (allogeneic) BM derived DCs as described herein. Supernatants were tested for IL-2 and TNF-α by ELISA. There was no significant difference in the cytokine secretion by WT or miR-155−/− T cells on allogeneic stimulation (n=4).

FIG. 13D. In vivo proliferation assays of WT or miR-155^{-/-} donor T cells in lethally irradiated recipients. Three days post transplant, spleens were harvested and CFSE divisions were evaluated on gated donor H2 Kd− CD4⁺/CD8⁺ cells. A representative donor CD4⁺ and CD8⁺ CFSE histogram from each group is shown in the left.

FIG. 13E. Precursor frequency was calculated and shown in the right panel (n=5 per group). Data was reproduced in another experiment at day 4.

FIG. 13F. Homeostatic proliferation profiles of WT or miR-155 deficient donor T cells in congenic lethally irradiated B6 recipients. Congenic lethally irradiated B6 CD45.1 mice received BM cells as well as CFSE labeled CD45.2 miR-155^{+/+} or miR-155^{-/-} splenocytes. Three days later, spleens were harvested and the precursor frequency was determined on the donor CD45.2 CD4⁺/CD8⁺ T cells (n=5).

FIGS. 14A-14B. Treatment with LNA-antimiR-155 Decreases the Severity of aGVHD and Prolongs the Survival of MHC-Mismatched Recipient Mice.

Lethally irradiated recipient F1 mice transplanted with BM (5×10⁶)+B6 WT spleen cells (20×10⁶) were treated with LNA-antimiR-155 (n=10) or control LNA oligos (n=10) starting at day 7 with a loading dose 25 mg/kg followed by 5 mg/Kg I.V. twice weekly up to day 30 after infusion of donor B6 splenocytes.

FIG. 14A. Clinical GVHD scores post transplant from lethally irradiated F1 recipients transplanted with B6 donor BM and splenocytes and treated with LNA-antimiR-155 or control oligonucleotide. The P values were obtained by using t-test.

FIG. 14B. Survival rate of the different recipient F1 mice groups. Comparisons between survival curves were performed using log-rank test.

FIG. 15. Histopathological assessment of CD3+ staining in human small and large bowel samples from patients with aGVHD and high miR-155 expression. Anti CD3 immunostains were performed using mouse monoclonal anti-human CD3 Ab (DAKO) (right panel). Hematoxylin and eosin stains are shown in the left panel. The two first panels represent one case, different sections at 200× and 400×. The last two panels represent a second case, two different sections at 200× and 400×. In both cases and in H&E the colon tissue shows marked inflammation with a loss of glands and concomitant erosion. Note the many inflammatory cells in the lamina propria were positive for CD3+ staining (Arrows).

FIGS. 16A-16B. The purity of the isolated T cells was greater than 95% by FACS using CD3 as the marker.

FIG. 17. Table 1. MicroRNAs differentially expressed between CD4+CD62L− cells from mice from mice with aGVHD (BM+splenocytes) compared with controls (BM alone).

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

### Terms

It is to be understood that both the foregoing general description and the following detailed description are exemplary and explanatory only and are not intended to limit the scope of the current teachings. In this application, the use of the singular includes the plural unless specifically stated otherwise. In order to facilitate review of the various embodiments of the disclosure, the following explanations of specific terms are provided.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The use of the word “a” or “an” when used in conjunction with the term “comprising” in the claims and/or the specification may mean “one,” but it is also consistent with the meaning of “one or more,” “at least one,” and “one or more than one.”

Also, the use of “comprise”, “contain”, and “include”, or modifications of those root words, for example but not limited to, “comprises”, “contained”, and “including”, are not intended to be limiting. The term “and/or” means that the terms before and after can be taken together or separately. For illustration purposes, but not as a limitation, “X and/or Y” can mean “X” or “Y” or “X and Y”.

It is understood that a miRNA is derived from genomic sequences or a gene. In this respect, the term “gene” is used for simplicity to refer to the genomic sequence encoding the precursor miRNA for a given miRNA. However, embodiments of the invention may involve genomic sequences of a miRNA that are involved in its expression, such as a promoter or other regulatory sequences.

The terms “miR,” “mir” and “miRNA” generally refer to microRNA, a class of small RNA molecules that are capable of modulating RNA translation (see, Zeng and Cullen, RNA, 9(1):112-123, 2003; Kidner and Martienssen Trends Genet, 19(1):13-6, 2003; Dennis C, Nature, 420(6917):732, 2002; Couzin J, Science 298(5602):2296-7, 2002, each of which is incorporated by reference herein).

MicroRNAs are generally 21-23 nucleotides in length. MicroRNAs are processed from primary transcripts known as pri-miRNA to short stem-loop structures called precursor (pre)-miRNA and finally to functional, mature microRNA. Mature microRNA molecules are partially complementary to one or more messenger RNA molecules, and their primary function is to down-regulate gene expression. MicroRNAs regulate gene expression through the RNAi pathway.

“miRNA nucleic acid” generally refers to RNA or DNA that encodes a miR as defined above, or is complementary to a nucleic acid sequence encoding a miR, or hybridizes to such RNA or DNA and remains stably bound to it under appropriate stringency conditions. Particularly included are genomic DNA, cDNA, mRNA, miRNA and antisense molecules, pri-miRNA, pre-miRNA, mature miRNA and miRNA seed sequences. Also included are nucleic acids based on alternative backbones or including alternative bases. mRNA nucleic acids can be derived from natural sources or synthesized.

It is to be understood that a miRNAs or pre-miRNAs can be 18-100 nucleotides in length, and more preferably from 18-80 nucleotides in length. For example, mature miRNAs can have a length of 19-30 nucleotides, preferably 21-25 nucleotides, particularly 21, 22, 23, 24, or 25 nucleotides. MicroRNA precursors typically have a length of about 70-100 nucleotides and have a hairpin conformation. Thus, once a sequence of a miRNA or a pre-miRNA is known, a miRNA antagonist that is sufficiently complementary to a portion of the miRNA or the pre-miRNA can be designed according to the rules of Watson and Crick base pairing. As used herein, the term “sufficiently complementary” generally means that two sequences are sufficiently complementary such that a duplex can be formed between them under physiologic conditions. A miRNA antagonist sequence that is sufficiently complementary to a miRNA or pre-miRNA target sequence can be 70%, 80%, 90%, or more identical to the miRNA or pre-miRNA sequence. In one embodiment, the miRNA antagonist contains no more than 1, 2 or 3 nucleotides that are not complementary to the miRNA or pre-miRNA target sequence. In another embodiment, the miRNA antagonist is 100% complementary to a miRNA or pre-miRNA target sequence. Sequences for miRNAs are available publicly through the miRBase registry (Griffiths-Jones, et al., Nucleic Acids Res., 36(Database Issue):D154-D158 (2008); Griffiths-Jones, et al., Nucleic Acids Res., 36(Database Issue):D140-D144 (2008); Griffiths-Jones, et al., Nucleic Acids Res., 36(Database Issue):D109-D111 (2008)).

The term “miRNA” generally refers to a single-stranded molecule, but in specific embodiments, molecules can encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary miRNA probes of embodiments of the invention can be or be at least 60, 65, 70, 75, 80, 85, 90, 95, or 100% (and all ranges inbetween) complementary to their target.

“MicroRNA seed sequence,” “miRNA seed sequence,” “seed region” and “seed portion” generally refer to nucleotides 2-7 or 2-8 of the mature miRNA sequence. The miRNA seed sequence is typically located at the 5′ end of the miRNA.

A “miR-specific inhibitor” may be an anti-miRNA (anti-miR) oligonucleotide. Anti-miRNAs may be single stranded molecules. Anti-miRs may comprise RNA or DNA or have non-nucleotide components. Anti-miRs anneal with and block mature microRNAs through extensive sequence complementarity. In some embodiments, an anti-miR may comprise a nucleotide sequence that is a perfect complement of the entire miRNA. In some embodiments, an anti-miR comprises a nucleotide sequence of at least 6 consecutive nucleotides that are complementary to the seed region of a microRNA molecule at positions 2-8 and has at least 50%, 60%, 70%, 80%, or 90% complementarity to the rest of the miRNA. In other embodiments, the anti-miR may comprise additional flanking sequence, complimentary to adjacent primary (pri-miRNA) sequences. Chemical modifications, such as 2′-O-methyl; locked nucleic acids (LNA); and 2′-O-methyl, phosphorothioate, cholesterol (antagomir); 2′-O-methoxyethyl can be used. Chemically modified anti-miRs are commercially available from a variety of sources, including but not limited to Sigma-Proligo, Ambion, Exiqon, and Dharmacon.

The miRNA antagonists can be oligomers or polymers of RNA or DNA, and can contain modifications to their nucleobases, sugar groups, phosphate groups, or covalent internucleoside linkages. In certain embodiment, modifications include those that increase the stability of the miRNA antagonists or enhance cellular uptake of the miRNA antagonists. In one embodiment, the miRNA antagonists are antagomirs, which have 2′-O-methylation of the sugars, a phosphorothioate backbone and a terminal cholesterol moiety.

In some embodiments, miR-specific inhibitors possess at least one microRNA binding site, mimicking the microRNA target (target mimics). These target mimics may possess at least one nucleotide sequence comprising 6 consecutive nucleotides complementary to positions 2-8 of the miRNA seed region. Alternatively, these target mimics may comprise a nucleotide sequence with complementarity to the entire miRNA. These target mimics may be vector encoded. Vector encoded target mimics may have one or more microRNA binding sites in the 5′ or 3′ UTR of a reporter gene. The target mimics may possess microRNA binding sites for more than one microRNA family. The microRNA binding site of the target mimic may be designed to mismatch positions 9-12 of the microRNA to prevent miRNA-guided cleavage of the target mimic.

In an alternative embodiment, a miR-specific inhibitor may interact with the miRNA binding site in a target transcript, preventing its interaction with a miRNA.

The terms “miRNA specific inhibitor” and “miRNA antagonist,” generally refer to an agent that reduces or inhibits the expression, stability, or activity of a miRNA (e.g., miR-155). A miRNA antagonist may function, for example, by blocking the activity of a miRNA (e.g., blocking the ability of a miRNA to function as a translational repressor and/or activator of one or more miRNA targets), or by mediating miRNA degradation. Exemplary miRNA antagonists include nucleic acids, for example, antisense locked nucleic acid molecules (LNAs), antagomirs, or 2′O-methyl antisense RNAs targeting a miRNA.

The phrase “inhibiting expression of a target gene” generally refers to the ability of an RNAi agent, such as a siRNA, to silence, reduce, or inhibit expression of a target gene. The another way, to “inhibit”, “down-regulate”, or “reduce”, it is meant that the expression of the gene, or level of RNA molecules or equivalent RNA molecules encoding one or more proteins or protein subunits, or activity of one or more proteins or protein subunits, is reduced below that observed in the absence of the RNAi agent.

For example, in one embodiment, inhibition, down-regulation, or reduction contemplates inhibition of the target mRNA below the level observed in the presence of, for example, a siRNA molecule with scrambled sequence or with mismatches.

To examine the extent of gene silencing, a test sample (e.g., a biological sample from organism of interest expressing the target gene(s) or a sample of cells in culture expressing the target gene(s)) is contacted with a siRNA that silences, reduces, or inhibits expression of the target gene(s). Expression of the target gene in the test sample is compared to expression of the target gene in a control sample (e.g., a biological sample from organism of interest expressing the target gene or a sample of cells in culture expressing the target gene) that is not contacted with the siRNA. Control samples (i.e., samples expressing the target gene) are assigned a value of 100%. Silencing, inhibition, or reduction of expression of a target gene is achieved when the value of the test sample relative to the control sample is about 95%, 90%, 85%, 80%, 75%, 70%, 65%, 60%, 55%, 50%, 45%, 40%, 35%, 30%, 25%, 20%, 10% or 0%. Suitable assays include, e.g., examination of protein or mRNA levels using techniques known to those of skill in the art such as dot blots, northern blots, in situ hybridization, ELISA, microarray hybridization, immunoprecipitation, enzyme function, as well as phenotypic assays known to those of skill in the art.

An “effective amount” or “therapeutically effective amount” of a miR-specific inhibitor is an amount sufficient to produce the desired effect, e.g., inhibition of expression of a target sequence in comparison to the normal expression level detected in the absence of the miR-specific inhibitor. Inhibition of expression of a target gene or target sequence by a miR-specific inhibitor is achieved when the expression level of the target gene mRNA or protein is about 90%, 80%, 70%, 60%, 50%, 40%, 30%, 25%, 20%, 15%, 10%, 5%, or 0% relative to the expression level of the target gene mRNA or protein of a control sample. The desired effect of a miR-specific inhibitor may also be measured by detecting an increase in the expression of genes down-regulated by the miRNA targeted by the miR-specific inhibitor.

By “modulate” is meant that the expression of the gene, or level of RNA molecule or equivalent RNA molecules encoding one or more proteins or protein subunits, or activity of one or more proteins or protein subunits is up-regulated or down-regulated, such that expression, level, or activity is greater than or less than that observed in the absence of the modulator. For example, the term “modulate” can mean “inhibit,” but the use of the word “modulate” is not limited to this definition.

Non-limiting examples of suitable sequence variants of miRNA can include: substitutional, insertional or deletional variants. Insertions include 5′ and/or 3′ terminal fusions as well as intrasequence insertions of single or multiple residues. Insertions can also be introduced within the mature sequence. These, however, can be smaller insertions than those at the 5′ or 3′ terminus, on the order of 1 to 4 residues, preferably 2 residues, most preferably 1 residue.

Insertional sequence variants of miRNA are those in which one or more residues are introduced into a predetermined site in the target miRNA. Most commonly insertional variants are fusions of nucleic acids at the 5′ or 3′ terminus of the miRNA.

Deletion variants are characterized by the removal of one or more residues from the miRNA sequence. These variants ordinarily are prepared by site specific mutagenesis of nucleotides in the DNA encoding miRNA, thereby producing DNA encoding the variant, and thereafter expressing the DNA in recombinant cell culture. However, variant miRNA fragments may be conveniently prepared by in vitro synthesis. The variants typically exhibit the same qualitative biological activity as the naturally-occurring analogue, although variants also are selected in order to modify the characteristics of miRNA.

Substitutional variants are those in which at least one residue sequence has been removed and a different residue inserted in its place. While the site for introducing a sequence variation is predetermined, the mutation per se need not be predetermined. For example, in order to optimize the performance of a mutation at a given site, random mutagenesis may be conducted at the target region and the expressed miRNA variants screened for the optimal combination of desired activity. Various suitable techniques for making substitution mutations at predetermined sites in DNA having a known sequence can be used.

Nucleotide substitutions are typically of single residues; insertions usually will be on the order of about from 1 to 10 residues; and deletions will range about from 1 to 30 residues. Deletions or insertions preferably are made in adjacent pairs; i.e., a deletion of 2 residues or insertion of 2 residues. Substitutions, deletion, insertions or any combination thereof may be combined to arrive at a final construct.

Changes may be made to decrease the activity of the miRNA, and all such modifications to the nucleotide sequences encoding such miRNA are encompassed.

An “isolated nucleic acid or DNA” is generally understood to mean chemically synthesized DNA, cDNA or genomic DNA with or without the 3′ and/or 5′ flanking regions. DNA encoding miRNA can be obtained from other sources by, for example” a) obtaining a cDNA library from cells containing mRNA; b) conducting hybridization analysis with labeled DNA encoding miRNA or fragments thereof in order to detect clones in the cDNA library containing homologous sequences; and, c) analyzing the clones by restriction enzyme analysis and nucleic acid sequencing to identify full-length clones.

As used herein nucleic acids and/or nucleic acid sequences are “homologous” when they are derived, naturally or artificially, from a common ancestral nucleic acid or nucleic acid sequence. Homology is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence similarity percentages (e.g., BLASTN using default parameters) are generally available. Software for performing BLAST analyses is publicly available through the National Center for Biotechnology Information.

The term “detecting the level of miR expression” generally refers to quantifying the amount of such miR present in a sample. Detecting expression of a miR, or any microRNA, can be achieved using any method known in the art or described herein, such as by qRT-PCR. Detecting expression of a miR includes detecting expression of either a mature form of the miR or a precursor form that is correlated with the miR expression. For example, miRNA detection methods involve sequence specific detection, such as by RT-PCR. miR-specific primers and probes can be designed using the precursor and mature miR nucleic acid sequences, which are known in the art and include modifications which do not change the function of the sequences.

The terms “low miR-expression” and “high miR-expression” are relative terms that refer to the level of miR/s found in a sample. In some embodiments, low miR- and high miR-expression are determined by comparison of miR/s levels in a group of test samples and control samples. Low and high expression can then be assigned to each sample based on whether the expression of a miR in a sample is above (high) or below (low) the average or median miR expression level. For individual samples, high or low miR expression can be determined by comparison of the sample to a control or reference sample known to have high or low expression, or by comparison to a standard value. Low and high miR expression can include expression of either the precursor or mature forms of miR, or both.

The term “expression vector” generally refers to a nucleic acid construct that can be generated recombinantly or synthetically. An expression vector generally includes a series of specified nucleic acid elements that enable transcription of a particular gene in a host cell. Generally, the gene expression is placed under the control of certain regulatory elements, such as constitutive or inducible promoters.

The term “operably linked” is used to describe the connection between regulatory elements and a gene or its coding region. That is, gene expression is typically placed under the control of certain regulatory elements, for example, without limitation, constitutive or inducible promoters, tissue-specific regulatory elements, and enhancers. A gene or coding region is the to be “operably linked to” or “operatively linked to” or “operably associated with” the regulatory elements, meaning that the gene or coding region is controlled or influenced by the regulatory element.

The term “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB.

The terms “agent” and “drug” generally refer to any therapeutic agents (e.g., chemotherapeutic compounds and/or molecular therapeutic compounds), antisense therapies, radiation therapies, or surgical interventions, used in the treatment of a particular disease or disorder.

The term “adjunctive therapy” generally refers to a treatment used in combination with a primary treatment to improve the effects of the primary treatment.

The term “clinical outcome” generally refers to the health status of a subject following treatment for a disease or disorder, or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

The term “decrease in survival” generally refers to a decrease in the length of time before death of a subject, or an increase in the risk of death for the subject.

The term “control” generally refers to a sample or standard used for comparison with an experimental sample, such as a sample obtained from a subject. In some embodiments, the control is a sample obtained from a healthy subject. In some embodiments, the control is cell/tissue sample obtained from the same subject. In some embodiments, the control is a historical control or standard value (i.e., a previously tested control sample or group of samples that represent baseline or normal values, such as the level in a control sample). In other embodiments, the control is a sample obtained from a healthy subject, such as a donor. Test samples and control samples can be obtained according to any method known in the art.

The term “cytokines” generally refers to proteins produced by a wide variety of hematopoietic and non-hematopoietic cells that affect the behavior of other cells. Cytokines are important for both the innate and adaptive immune responses.

The terms “prevent,” “preventing” and “prevention” generally refer to a decrease in the occurrence of disease or disorder in a subject. The prevention may be complete, e.g., the total absence of the disease or disorder in the subject. The prevention may also be partial, such that the occurrence of the disease or disorder in the subject is less than that which would have occurred without embodiments of the present invention. “Preventing” a disease generally refers to inhibiting the full development of a disease.

The terms “treating” and/or “ameliorating a disease” generally refer to a therapeutic intervention that ameliorates a sign or symptom of a disease or disorder after it has begun to develop. “Ameliorating” generally refers to the reduction in the number or severity of signs or symptoms of a disease or disorder.

The term “subject” includes human and non-human animals. The preferred subject for treatment is a human. “Subject” and “subject” are used interchangeably herein.

The term “therapeutic” generally is a generic term that includes both diagnosis and treatment.

The term “therapeutic agent” generally refers to a chemical compound, small molecule, or other composition, such as an antisense compound, protein, peptide, small molecule, nucleic acid, antibody, protease inhibitor, hormone, chemokine or cytokine, capable of inducing a desired therapeutic or prophylactic effect when properly administered to a subject. Multiple therapeutic agents may be used simultaneously or in sequence. For example, with respect to the treatment of graft-versus-host disease (GVHD), in one embodiment, a therapeutic agent could be selected from immunosuppressive treatment, corticosteroids, calcineurin inhibitors, methotrexate, cyclosporine, antithymocyte globulin, prednisolone, methylprednisolone, extracorporeal photopheresis, anti-tumour necrosis factor α antibodies, etanercept, infliximab, mammalian target of rapamycin (mTOR) inhibitors, sirolimus, mycophenolate mofetil, interleukin-2 receptor antibodies, alemtuzumab, pentostatin, and antihistamines.

As used herein, a “candidate agent” is a compound selected for screening to determine if it can function as a therapeutic agent. “Incubating” includes a sufficient amount of time for an agent to interact with a cell or tissue. “Contacting” includes incubating an agent in solid or in liquid form with a cell or tissue. “Treating” a cell or tissue with an agent includes contacting or incubating the agent with the cell or tissue.

The term “therapeutically effective amount” generally refers to that amount of the therapeutic agent sufficient to result in amelioration of one or more symptoms of a disorder, or prevent advancement of a disorder, or cause regression of the disease or disorder. For example, with respect to the treatment of graft-versus-host disease (GVHD), in one embodiment, a therapeutically effective amount will refer to the amount of a therapeutic agent that decreases the rate of rejection, or increases survival time by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

A “therapeutically effective amount” can be a quantity of a specified pharmaceutical or therapeutic agent sufficient to achieve a desired effect in a subject, or in a cell, being treated with the agent. For example, this can be the amount of a therapeutic agent that alters the expression of miR/s, and thereby prevents, treats or ameliorates the disease or disorder in a subject. The effective amount of the agent will be dependent on several factors, including, but not limited to the subject or cells being treated, and the manner of administration of the therapeutic composition.

The term “pharmaceutically acceptable vehicles” generally refers to such pharmaceutically acceptable carriers (vehicles) as would be generally used. Remington's Pharmaceutical Sciences, by E. W. Martin, Mack Publishing Co., Easton, Pa., 20 Edition, describes compositions and formulations suitable for pharmaceutical delivery of one or more therapeutic compounds, molecules or agents. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The term “pharmaceutically acceptable salt” generally refers to any salt (e.g., obtained by reaction with an acid or a base) of a compound of embodiments of the present invention that is physiologically tolerated in the target animal (e.g., a mammal). Salts of the compounds of embodiments of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of embodiments of the invention and their pharmaceutically acceptable acid addition salts. Examples of bases include, but are not limited to, alkali metal (e.g., sodium) hydroxides, alkaline earth metal (e.g., magnesium) hydroxides, ammonia, and the like. Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, mesylate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of embodiments of the present invention compounded with a suitable cation such as Na+, NH4+, and NW4+ (wherein W is a C1-4 alkyl group), and the like. For therapeutic use, salts of the compounds of embodiments of the present invention are contemplated as being pharmaceutically acceptable. However, salts of acids and bases that are non-pharmaceutically acceptable may also find use, for example, in the preparation or purification of a pharmaceutically acceptable compound

miR-155 Specific Inhibitors and miR-155 Antagonists

The terms “miRNA-155” and “miR-155” are used interchangeably and, unless otherwise indicated, refer to microRNA-155, including miR-155, pri-miR-155, pre-miR-155, mature miR-155, miRNA-155 seed sequence, sequences comprising a miRNA-155 seed sequence, and variants thereof.

In some embodiments, nucleic acids are used that are capable of blocking the activity of a miRNA (anti-miRNA or anti-miR). Such nucleic acids include, for example, antisense miR-155. For example, a “miR-155 antagonist” means an agent designed to interfere with or inhibit the activity of miRNA-155.

In certain embodiments, the miR-155 antagonist can be comprised of an antisense compound targeted to a miRNA. For example, the miR-155 antagonist can be comprised of a small molecule, or the like that interferes with or inhibits the activity of a miRNA.

In certain embodiments, the miR-155 antagonist can be comprised of a modified oligonucleotide having a nucleobase sequence that is complementary to the nucleobase sequence of a miRNA, or a precursor thereof.

In certain embodiments, the anti-miR is an antisense miR-155 nucleic acid comprising a total of about 5 to about 100 or more, more preferably about 10 to about 60 nucleotides, and has a sequence that is preferably complementary to at least the seed region of miR-155. In one embodiment, an anti-miRNA may comprise a total of at least about 5, to about 26 nucleotides. In some embodiments, the sequence of the anti-miRNA can comprise at least 5 nucleotides that are substantially complementary to the 5′ region of a miR-155, at least 5 nucleotides that are substantially complementary to the 3′ region of a miR-155, at least 4-7 nucleotides that are substantially complementary to a miR-155 seed sequence, or at least 5-12 nucleotide that are substantially complementary to the flanking regions of a miR-155 seed sequence.

In some embodiments, the anti-miR-155 comprises the complement of a sequence of the miR-155. In other embodiments an anti-miR-155 comprises the complement of the seed sequence or is able to hybridize under stringent conditions to the seed sequence. In certain embodiments, preferred molecules are those that are able to hybridize under stringent conditions to the complement of a cDNA encoding a mature miR-155.

It is to be understood that the methods described herein are not limited by the source of the miR-155 or anti-miR-155. The miR-155 can be from a human or non-human mammal, derived from any recombinant source, synthesized in vitro or by chemical synthesis. The nucleotide may be DNA or RNA and may exist in a double-stranded, single-stranded or partially double-stranded form, depending on the particular context.

miR-155 and anti-miR-155 nucleic acids may be prepared by any conventional means typically used to prepare nucleic acids in large quantity. For example, nucleic acids may be chemically synthesized using commercially available reagents and synthesizers by methods that are well-known in the art and/or using automated synthesis methods.

It is also be understood that the methods described herein are not limited to naturally occurring miR-155 sequences; rather, mutants and variants of miR-155 sequences are also within the contemplated scope. For example, nucleotide sequences that encode a mutant of a miR-155 that is a miR-155 with one or more substitutions, additions and/or deletions, and fragments of miR-155 as well as truncated versions of miR-155 maybe also be useful in the methods described herein.

It is also to be understood that, in certain embodiments, in order to increase the stability and/or optimize the delivery of the sense or antisense oligonucleotides, modified nucleotides or backbone modifications can be used. In some embodiments, a miR-155 or anti-miR-155 oligonucleotide can be modified to enhance delivery to target cells. Nucleic acid molecules encoding miR-155 and anti-miR-155 can be used in some embodiments to modulate function, activity and/or proliferation of immune cells.

In certain embodiments, the miR-155 antagonists can be single-stranded, double stranded, partially double stranded or hairpin structured oligonucleotides that include a nucleotide sequence sufficiently complementary to hybridize to a selected miR-155 or pre-miR-155 target sequence. As used herein, the term “partially double stranded” generally refers to double stranded structures that contain less nucleotides than the complementary strand. In general, partially double stranded oligonucleotides will have less than 75% double stranded structure, preferably less than 50%, and more preferably less than 25%, 20% or 15% double stranded structure.

In certain embodiments, the miRNA antagonist is sufficiently complementary to a portion of the miRNA or pre-miRNA sequence of a human miR-155. The miRNA antagonist can have a region that is at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to a portion of the miRNA or pre-miRNA sequence of a human miRNA.

Useful miRNA antagonists include oligonucleotides have at least 6, 7, 8, 9, 10, 11, 12, 13, 14, 15, 16, 17, 18, 19, 20 or more contiguous nucleotides substantially complementary to an endogenous miRNA or pre-miRNA that is overexpressed in subject having aGVHD as compared to subjects not having aGVHD. The disclosed miRNA antagonists preferably include a nucleotide sequence sufficiently complementary to hybridize to a miRNA target sequence of about 12 to 25 nucleotides, preferably about 15 to 23 nucleotides. In some embodiments, there will be nucleotide mismatches in the region of complementarity. In certain embodiment, the region of complementarily will have no more than 1, 2, 3, 4, or 5 mismatches.

In some embodiments, the miRNA antagonist is “exactly complementary” to has-miR-155. For example, hsa-miR155 has miRBase Accession No. MI0000681; hsa-miR-155-5p has Accession No. MIMAT0000646; and, hsa-miR1-55-3p has Accession No. MIMAT0004658. Thus, in one embodiment, the miRNA antagonist can anneal to the miRNA to form a hybrid made exclusively of Watson-Crick base pairs in the region of exact complementarity. Thus, in some embodiments, the miRNA antagonist specifically discriminates a single-nucleotide difference. In such cases, the miRNA antagonist only inhibits miRNA activity if exact complementarity is found in the region of the single-nucleotide difference. Also, in certain embodiments, the miRNA antagonists are oligomers or polymers of ribonucleic acid (RNA) or deoxyribonucleic acid (DNA) or modifications thereof. miRNA antagonists include oligonucleotides that contain naturally-occurring nucleobases, sugars and covalent internucleoside (backbone) linkages.

Delivery of Oligonucleotides and Expression Vectors to a Target Cell or Tissue

Expression vectors that contain anti-miR-155 coding sequence can be used to deliver an anti-miR155 to target cells. In certain embodiments, expression vectors can contain an anti-miR-155 sequence, optionally associated with a regulatory element that directs the expression of the coding sequence in a target cell. It is to be understood that the selection of particular vectors and/or expression control sequences to which the encoding sequence is operably linked generally depends (as is understood by those skilled in the art) on the particular functional properties desired; for example, the host cell to be transformed.

It is also to be understood that vectors useful with the methods described herein are preferably capable of directing replication in an appropriate host and of expression of the anti-miR-155 in a target cell, tissue or organ.

It is also to be understood that a useful vector can include a selection gene whose expression confers a detectable marker such as a drug resistance. Non-limiting examples of selection genes include those vectors that encode proteins that confer resistance to antibiotics or other toxins, e.g., ampicillin, neomycin, methotrexate, or tetracycline, complement auxotrophic deficiencies, or supply critical nutrients withheld from the media. It is also to be understood that the detectable marker can optionally be present on a separate plasmid and introduced by co-transfection.

It is also to be understood that expression control elements can be used to regulate the expression of an operably linked coding sequence. Non-limiting examples include: inducible promoters, constitutive promoters, enhancers, and other regulatory elements. In some embodiments an inducible promoter is used that is readily controlled, such as being responsive to a nutrient in the target cell's medium. In some embodiments, the promoter is the U6 promoter or CMV promoter. It is also to be understood that other methods, vectors, and target cells suitable for adaptation to the expression of miR-155 specific inhibitors in target cells can be readily adapted to the specific circumstances.

In certain embodiments, the anti-miR-155 oligonucleotide is delivered to a target cell. In other embodiments, an expression vector encoding the anti-miR-155 is delivered to a target cell where the anti-miR-155 is expressed. It is to be understood that different methods for delivery of oligonucleotides and expression vectors to target cells can be used.

In certain embodiments, the target cells may be present in a host, such as in a mammal, or may be in culture outside of a host. The target cells may be present in a graft. Thus, the delivery of miR-155 or anti-miR-155 to target cells in vivo, ex vivo and in vitro can accomplished in a suitable manner. In certain embodiments, a miR-155 or anti-miR-155 oligonucleotide is delivered to a target organ or tissue.

In certain embodiments, the mutation of a cell can be modulated (e.g., suppressed) by administering a miRNA-155 or anti-miR-155 oligonucleotide to the B cells or T cells. The numbers and/or activity of the cells can be modulated by administering a miRNA-155 or anti-miR-155 oligonucleotide to the cancer cells or to pre-cancerous cells. In certain embodiments, the immune function and/or development of the cells can be modulated by delivering the anti-miR-155 to the cells.

It is to be understood that the delivery of oligonucleotides and/or expression vectors to a target cell can be accomplished using different methods. In certain embodiments, a transfection agent can be used. In general, a transfection agent (e.g., a transfection reagent and/or delivery vehicle) can be a compound or compounds that bind(s) to or complex(es) with oligonucleotides and polynucleotides, and enhances their entry into cells. Non-limiting examples of useful transfection reagents include: cationic liposomes and lipids, polyamines, calcium phosphate precipitates, polycations, histone proteins, polyethylenimine, polylysine, and polyampholyte complexes.

Another delivery method can include electroporating miRNA/s into a cell without inducing significant cell death. In addition, miRNAs can be transfected at different concentrations.

Non-limiting examples of useful reagents for delivery of miRNA, anti-miRNA and expression vectors include: protein and polymer complexes (polyplexes), lipids and liposomes (lipoplexes), combinations of polymers and lipids (lipopolyplexes), and multilayered and recharged particles. Transfection agents may also condense nucleic acids. Transfection agents may also be used to associate functional groups with a polynucleotide. Functional groups can include cell targeting moieties, cell receptor ligands, nuclear localization signals, compounds that enhance release of contents from endosomes or other intracellular vesicles (such as membrane active compounds), and other compounds that alter the behavior or interactions of the compound or complex to which they are attached (interaction modifiers).

In certain embodiments, miR-155 or anti-miR-155 nucleic acids and a transfection reagent can be delivered systematically such as by injection. In other embodiments, they may be injected into particular areas comprising target cells, such as particular organs, or an inflamed tissue. The skilled artisan will be able to select and use an appropriate system for delivering miRNA-155, anti-miRNA-155 or an expression vector to target cells in vivo, ex vivo and/or in vitro without undue experimentation.

In some embodiments, miR-155 levels in a transplant patient may me measured and/or monitored. For example, a blood sample of a patient may be subjected to laboratory assay, for instance a hybridization assay, to measure reaction to an allogenic graft based on the expression of miR-155. The result of such testing may be communicated to a caregiver to inform the prognosis and further treatment of the patient. In one embodiment, anti-miR-155 may be delivered by an automated system to a transplant patient presenting high measured expression of miR-155. In another embodiment, miR-155 levels are measured periodically or continuously and an audible or visual alarm is triggered if the measured miR-155 levels reach a high threshold. The threshold is determined and measured by any of several techniques, for example, it may be given in copies/μl plasma, copies/mg whole blood, by percent, or by fold change. Likewise, TNFα may be measured to assess aGVHD response.

Cancer Therapies and Tissue Transplantation

The earliest phase of acute GVHD is set into motion by the damage caused by the underlying disease and exacerbated by host conditioning regimens, such as radiation or chemotherapy. Damaged host tissues secrete proinflammatory cytokines, such as tumor necrosis factor α (TNF-α) and interleukin-1 (IL-1), which contribute to the “cytokine storm” increasing the expression of adhesion molecules, costimulatory molecules, major histocompatibility complex (MHC) antigens, and chemokine gradients that alert the residual host and the infused donor immune cells. These “danger signals” activate host tissue cells including antigen presenting cells (APCs). Damage to the gastrointestinal tract from the conditioning is particularly important in this process, because it allows for systemic translocation of lipopolysaccharide (LPS) that further enhances host APC activation.

In certain cancers, it is advantageous to the cancer therapy subject to provide an immunocompetent donor tissue transplant. For example, hematopoietic stem cells may be provided to a subject following radiation therapy and/or chemotherapy. A subject is commonly immunocompromised due to such treatments. The immunocompetent donor cells induce a graft versus tumor (GvT) response, wherein the donor cells attack remaining host cancer cells. This is also known as a graft versus leukemia (GVL) response. This response is suppressed by many common immunosuppressive methods of treating or preventing aGVHD. Mitigating aGVHD by administering a miR-155 specific inhibitor enables minimizing aGVHD while enabling GvT. The miR-155 inhibitor may be administered to the host, or the graft may be treated with a miR-155 inhibitor. In one embodiment, the donor cells comprise T cells. In one embodiment, the donor cells comprise, or are derived from, at least one of: T cells, hematopoietic stem cells, bone marrow, spleen, peripheral blood, umbilical cord blood, amniotic fluid, and dental pulp. In one embodiment, the cancer being treated is one or more of: leukemia, acute myeloid leukemia (AML), chronic lymphocytic leukemia (CLL), chronic myelogenous leukemia (CML), lymphoma, myeloma, and neuroblastoma.

General Description

Described herein are the effects of miR-155 overexpression in acute host-versus-graft disease (aGVHD). Also described are methods related to miR-155 regulation in donor cells during aGVHD and modulation of the development of this process.

Modulation of miR-155 is a useful therapy for the prevention or amelioration of aGVHD after hematopoietic stem cell transplant (HSCT).

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. The following examples are intended to illustrate certain preferred embodiments of the invention and should not be interpreted to limit the scope of the invention as defined in the claims, unless so specified.

The value of the present invention can thus be seen by reference to the Examples herein.

### EXAMPLES

### Materials and Methods

Mice

C57/BL/6(H2^{b}), (DBA/Ca)×C57BL/6) F1, B10.BR-H2^{k} and B6.Cg-miR-155^{tm1.1Rsky}/j mice were purchased from Jackson ImmunoResearch laboratories (Bar Harbor, Me.). Bic-deficient (bic^{m2/m2}) mice were obtained from Dr Tridandapani (OSU). The miR155 tm1.RskyJ was developed using a modified bacterial artificial chromosome (BAC) targeting vector to replace a 0.97 kb portion of exon 2 of the bic/mir-155 gene with an in-frame b-galactosidase (lacZ) reporter gene whereas in the miR-155-deficient^{m2/m2}, the miR-155 hairpin precursor within exon two of Bic was replaced by a PGK-neomycin-resistance cassette using the bacterial recombineering system.

For the development of the LCK-miR-155 transgenic mouse model a 318 base pairs DNA fragment containing the precursor sequence of mouse miR-155 was amplified from 129SvJ mouse DNA. The fragment was then cloned into the BamHI unique cloning site of a modified pUC-19 plasmid next to the LCK promoter. The transgene was linearized by digestion with NotI and injected into the male pronuclei of mouse zygotes. Pups where then screened by PCR for random insertion of the transgene. Fifteen founding lines were identified with three lines having high expression of the transgene. All lines were bred and expanded. All mice used were bred and maintained in an OSU animal care facility. A group of B6 and all B10.BR mice were bred and maintained at the University of Minnesota animal care facility. For all experiments mice were used between 8 and 12 weeks of age.

Acute GVHD Murine Models

Mice were transplanted under standard protocols approved by the University committee on Use and Care of Laboratory Animals at The Ohio State University (OSU). Only age and sex matched mice were used for transplant experiments. Briefly, 8-12 weeks recipient mice (F1) were irradiated with 1.1 Gy administered in two fractions to minimize toxicity. T-cell depleted bone marrow (BM) cells (BM)(5×10⁶) plus 20×10⁶ total splenocytes from B6 WT (miR-155^{+/+}) or B6 miR-155 deficient (Cg-miR-155^{tm1.1Rsky}/j from now on named miR-155^{-/-}) donors were administered via tail vein injection after the radiation. One group of mice did not receive any splenocytes (BM only group) and a second group did not receive any cells (Radiation only group). (For other experiments, this protocol was modified and instead of splenocytes, untouched T cells (2×10⁶) from B6 miR-155 WT or deficient mice were used). This parent to F1 model of aGVHD was chosen to eliminate host-versus-graft rejection responses in attempt to eliminate differential engraftment kinetics. In addition, this is a full or major MHC mismatched model, where aGVHD develops in response to class I, II HLA molecules, and is dependent on mainly CD4⁺ cells, although CD8⁺ cells could elicit additional pathology. There were no significant differences in the T cell dose among the splenocytes preparations from miR-155^{-/-} and the WT B6 mice.

A second MHC mismatched aGVHD murine model was performed at the University of Minnesota using university approved animal protocols. In this model, B10.BR recipients were lethally irradiated with 10 Gy and were divided in three groups: group 1 received only BM cells (10×10⁶), group 2 received B6 WT BM (10×10⁶), +unfractionated splenocytes (25×10⁶) from B6 WT mice and group 3 received WT B6 BM (10×10⁶), +unfractionated splenocytes (25×10⁶) from B6 miR-155^{-/-} mice.

Clinical and Histological Assessment of aGVHD

Recipient mice were weighed four times a week and monitored daily for clinical signs of aGVHD and survival. GVHD scores were performed according to Cooke K R, Kobzik L, Martin T R, et al. An experimental model of idiopathic pneumonia syndrome after bone marrow transplantation: I. The roles of minor H antigens and endotoxin. Blood. 1996; 88(8):3230-3239. Briefly, this scoring system incorporate five clinical parameters; weight loss, posture (hunching), activity, fur texture and skin integrity.

Individual mice were ear tagged and graded (in a scale from 0 to 10) three times a week by two researchers (one of them blinded). Mice who reached an aGVHD score of ≧7 were very sick and were sacrificed and their tissues harvested. GVHD was also assessed by detailed histopathology analysis of liver, spleen and gut tissues using a previously reported scoring system with a range of 0 (absence of signs of GVHD) to 4 (maximal GVHD damage). Two experienced pathologists read all the samples in a blinded fashion.

MicroRNA Expression Analysis

MiR-155 expression levels were detected using the single tube TaqMan murine miRNA assays (Applied Biosystems). Normalization was performed using sno135 expression levels. Comparative real-time qPCR was performed in triplicate, including no-template controls. Relative expression was calculated using the comparative Cₜ method (Livak K J, Schmittgen T D. Analysis of relative gene expression data using real-time quantitative PCR and the 2(-Delta Delta C(T)) Method. Methods. 2001; 25(4):402-408).

GVL Experiments

Briefly, 1×10⁴ firefly luciferase transduced P815 (H-2d) cells (a mastocytoma derived from a DBA/2 mouse) were injected intravenously into F1 recipients on day 0 along with BM and B6 miR155^{+/+} or miR155^{-/-} donor splenocytes. Control groups included BM and P815 cells (leukemia alone). Separate cohorts were used for imaging and survival. P815-induced leukemic death was defined by the occurrence of either macroscopic tumor nodules in liver or spleen or hindleg paralysis. GVHD death was defined by the absence of leukemia and the presence of clinical and pathological signs of GVHD. Luciferase transduced P815 mastocytoma cell line was used.

In Vivo Imaging

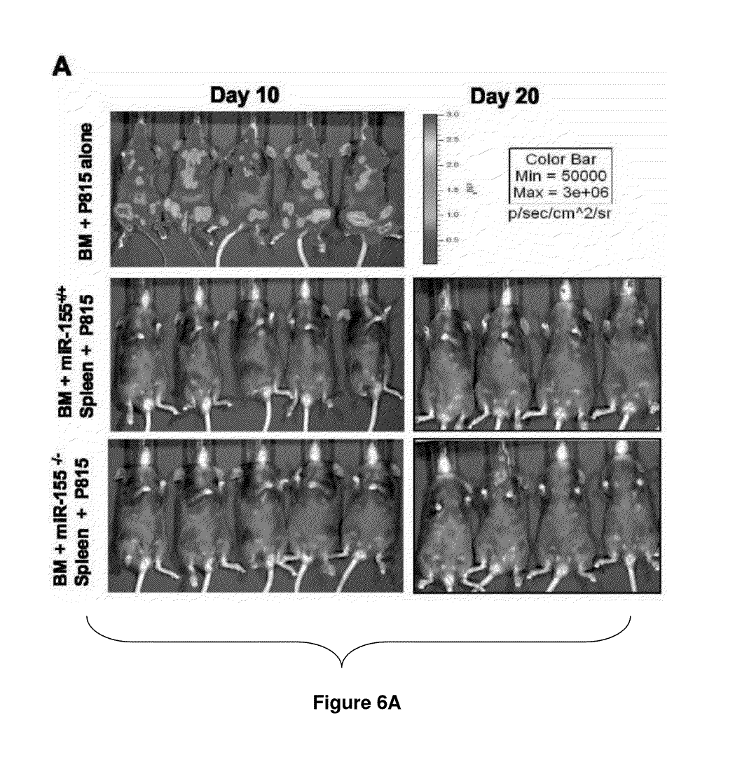

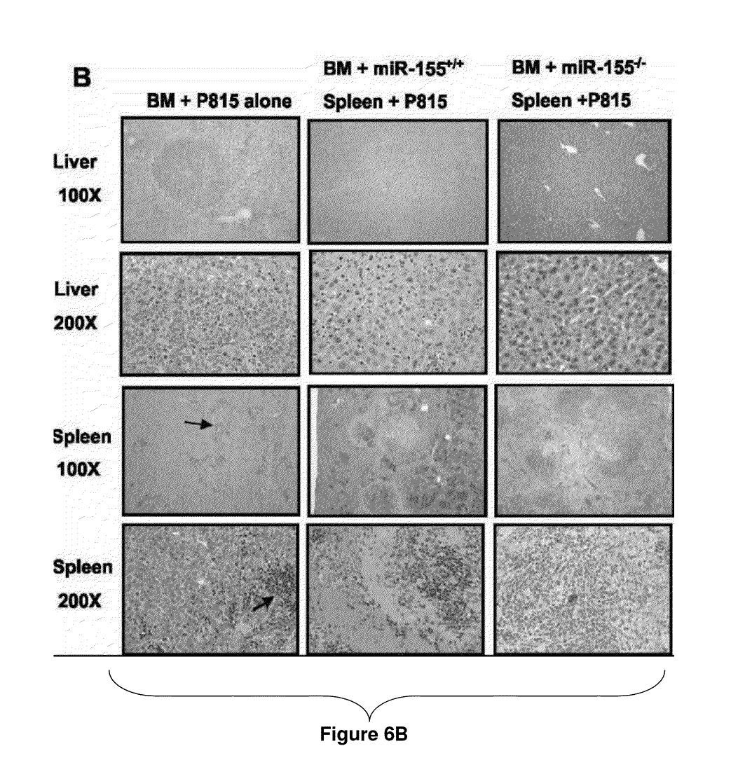

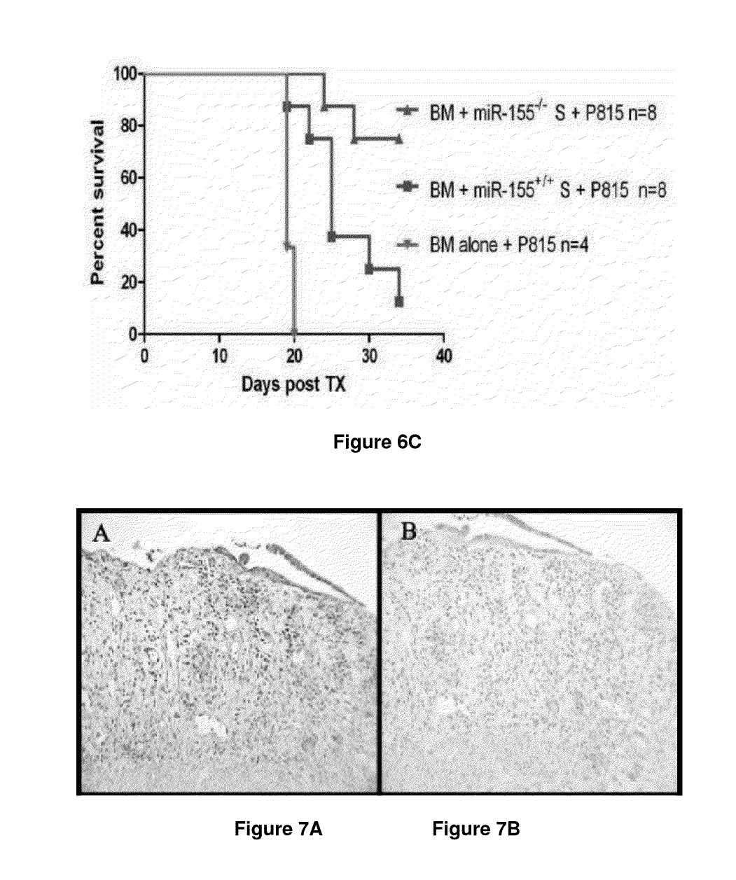

Xenogen IVIS imaging system (Caliper Life Sciences) was used for live animal imaging. Mice were anesthetized using isofluorane (Webster Veterinary). Firefly luciferin substrate (150 mg/kg body weight; 5 mg/mL in PBS; Caliper Life Sciences) was injected intraperitoneally and the IVIS imaging was performed 5 minutes after substrate injection. Whole body bioluminescent signal intensity was determined weekly and pseudocolor images overlaid on conventional photographs are shown in FIG. 6A. Data were analyzed and presented as photon counts per area.

Patient Samples

Histopathological samples of human gut aGVHD were obtained from 5 patients treated with allogeneic stem cell transplants at OSU. These samples were retrieved from the OSU pathology archives and the studies were approved by the institutional review board. In addition, three normal colon biopsies performed during screening for other diseases were used as controls.

In Vivo Treatment with LNA-antimiR-155 Oligonucleotides

The 8-mer seed-targeting LNA-antimiR-155 (5′-TAGCATTA-3′) and 8-mer LNA scramble control (5′-TCATACTA-3′) oligonucleotides were synthesized as unconjugated, fully LNA-substituted oligonucleotides with a complete PS backbone. Lethally irradiated recipient F1 mice transplanted with BM (5×10⁶)+B6 WT spleen cells (20×10⁶) were treated with LNA-antimiR-155 (n=10) or control LNA oligos (n=10) starting at day 7 with a loading dose 25 mg/kg followed by 5 mg/Kg I.V. twice weekly up to day 30 after infusion of donor B6 splenocytes. Clinical GVHD scores and survival was followed post-transplant.

Statistical Analysis

Survival data were analyzed using Kaplan Meier and long-rank test methods (SPSS and Graphma Prisma). Differences between continuous variables (e.g., cytokine expression and miRNA expression) were analyzed using t-tests. All P values are two sided.

Results

miR-155 Expression is Up-Regulated in Activated T Cells from Murine Recipients with aGVHD.

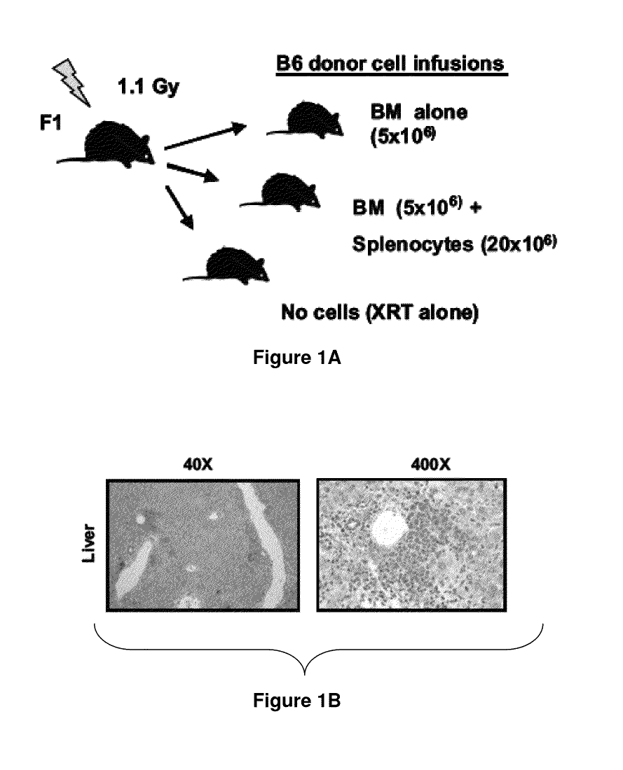

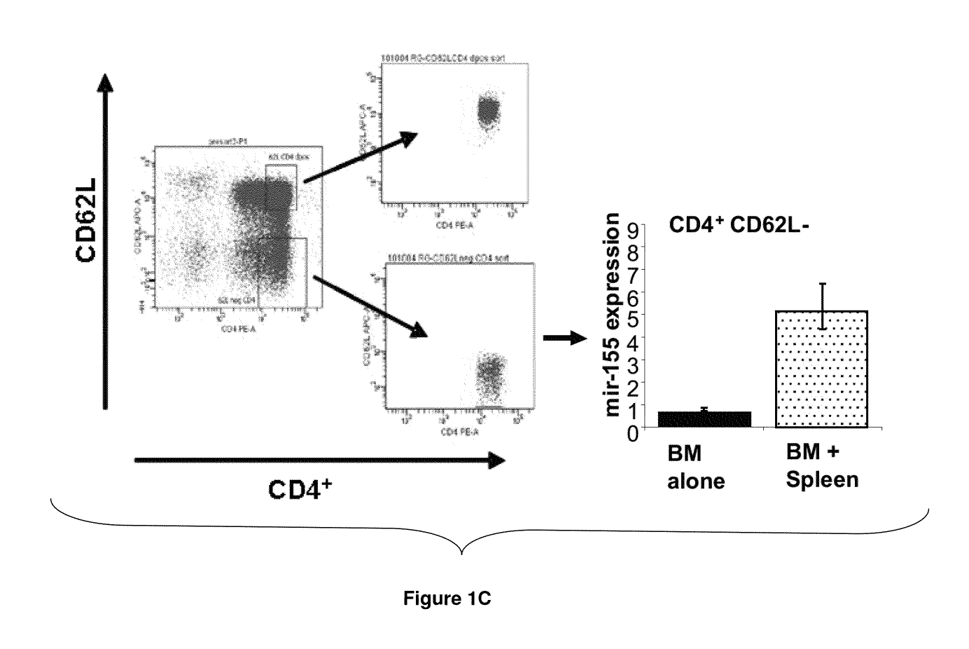

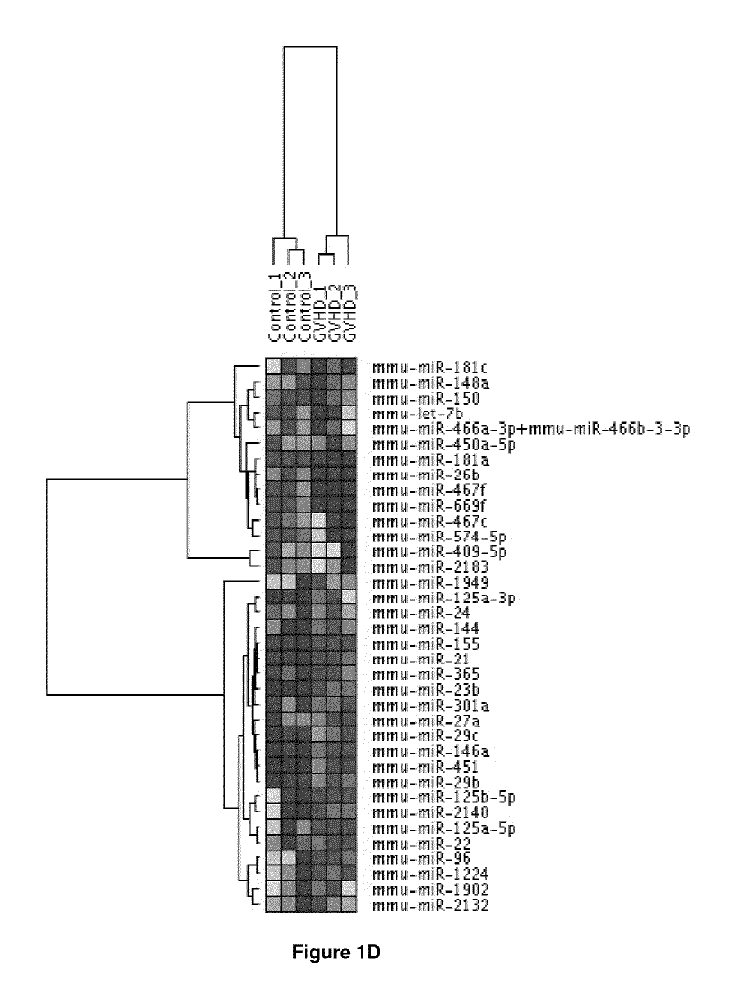

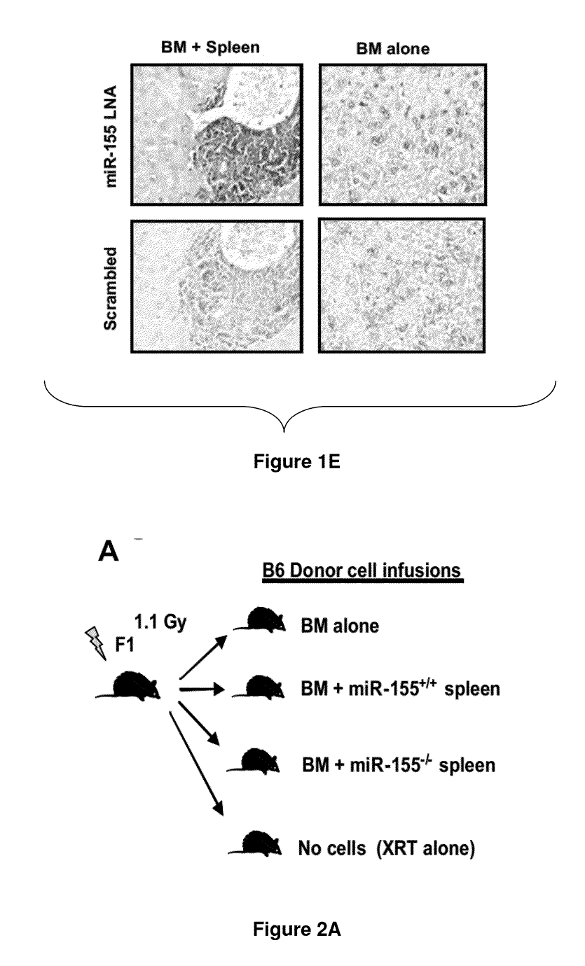

To determine whether miR-155 expression is up-regulated in T cell subsets during aGVHD, a major histocompatibility complex (MHC) mismatched HSCT model was used in which spleen cells (20×10⁶) and T cell depleted bone marrow (BM) (5×10⁶) from C57BL/6 (B6) donors were transferred intravenously (i.v.) into lethally irradiated B6D2F1 (F1) recipient mice (FIG. 1A).

Two additional groups were included as controls, with one group receiving no cell infusion (irradiation only), and a second group receiving only BM. This model of haplotype mismatched MHC (Class I and II) was chosen because the aGVHD that develops is primarily dependent on CD4⁺ T cells; most of the T cell alterations in miR-155 deficient mice are in CD4⁺ cells. However, CD8⁺ T cells also contribute to the development of aGVHD in this model due to class I and minor HLA disparities; thus, the expression of miR-155 can be determined in functionally important CD8⁺ subsets as well. Mice receiving donor BM plus spleen cells (n=3) developed severe aGVHD that was confirmed by liver histology (FIG. 1B).

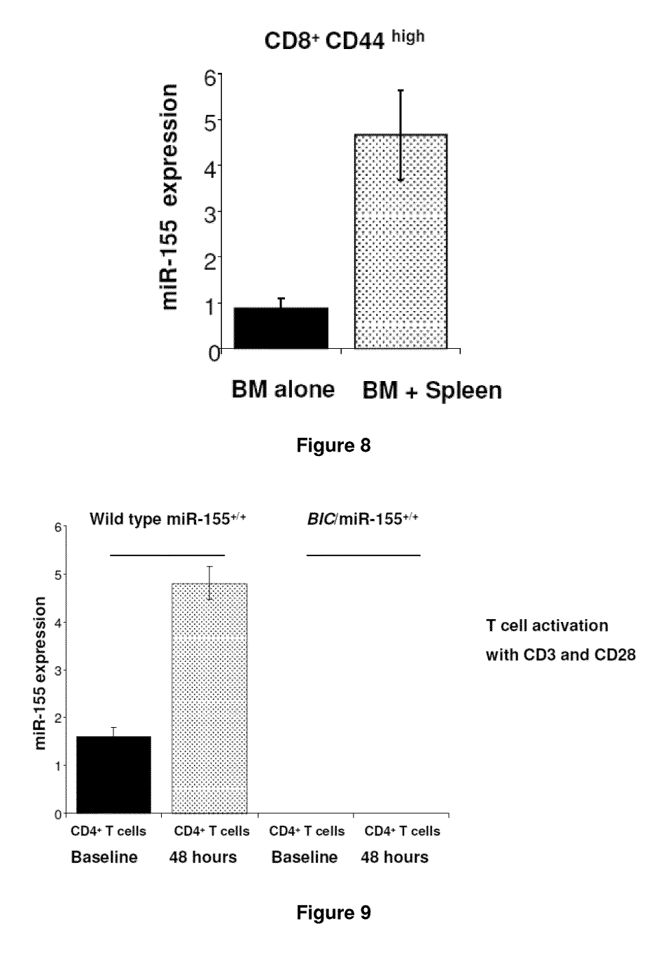

Mice were sacrificed when they achieved a clinical GVHD score of ≧7 (median time 21 days post BMT; range 19-23). Control mice treated with BM only were sacrificed at the same time point. CD4⁺CD62L⁻ (memory effectors) and CD8⁺CD44⁺(effectors active) cell subpopulation were isolated from the spleen of the sacrificed mice by using a combination of column magnetic bead and cell sorting. Total RNA was extracted from these highly purified cell populations (FIG. 1C and FIG. 8).

Both CD4⁺ and CD8⁺ cell populations isolated from mice with aGVHD exhibited increased miR-155 expression (6.5 and 5 fold, respectively) with respect to the same cell populations obtained from the controls (FIG. 1C and FIG. 8).

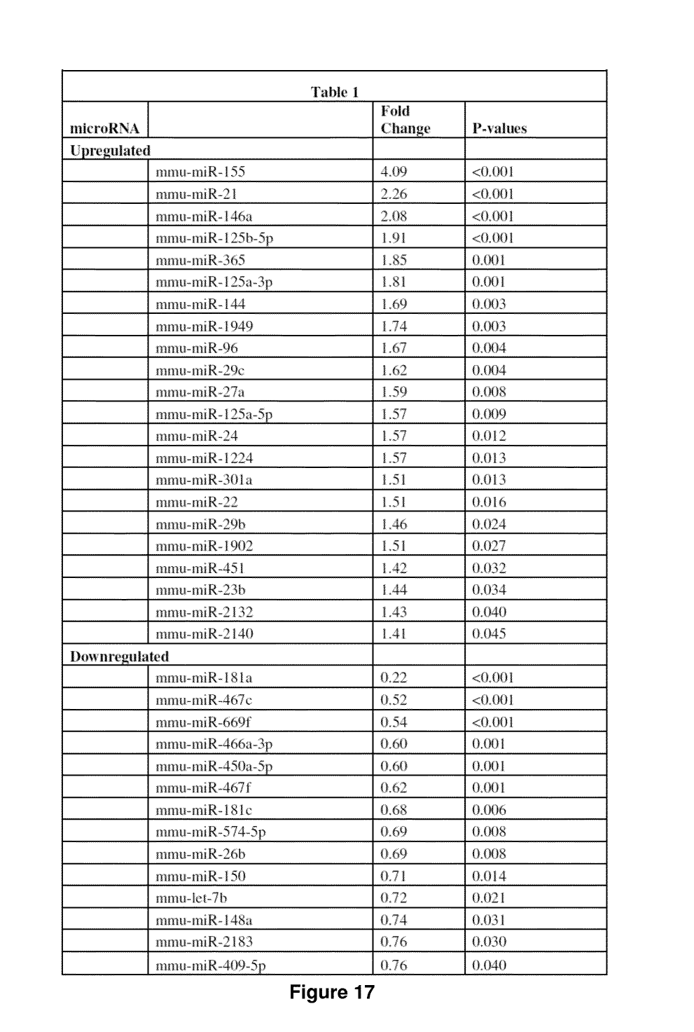

These results were validated in a second independent experiment using CD4⁺CD62L⁻ cells but using a different method of miRNA profiling (Nanostring). miR-155 was the top up-regulated miRNA in CD4⁺CD62L⁻ cells from mice with aGVHD vs controls (BM alone) (4 fold increase, P<0.001, FIG. 17—Table 1 and FIG. 1D.

The expression of miR-155 in the infiltrating lymphocytes of murine liver biopsies with aGVHD mice (n=3) or controls (BM alone n=3) using locked nucleic acid (LNA) based in situ hybridization was determined. A strong expression of miR-155 was observed within the lymphocyte infiltrates in the liver of all mice with aGVHD (FIG. 1E).

Recipients of miR-155 Deficient Splenocytes do not Develop Severe aGVHD and Show Increased Survival

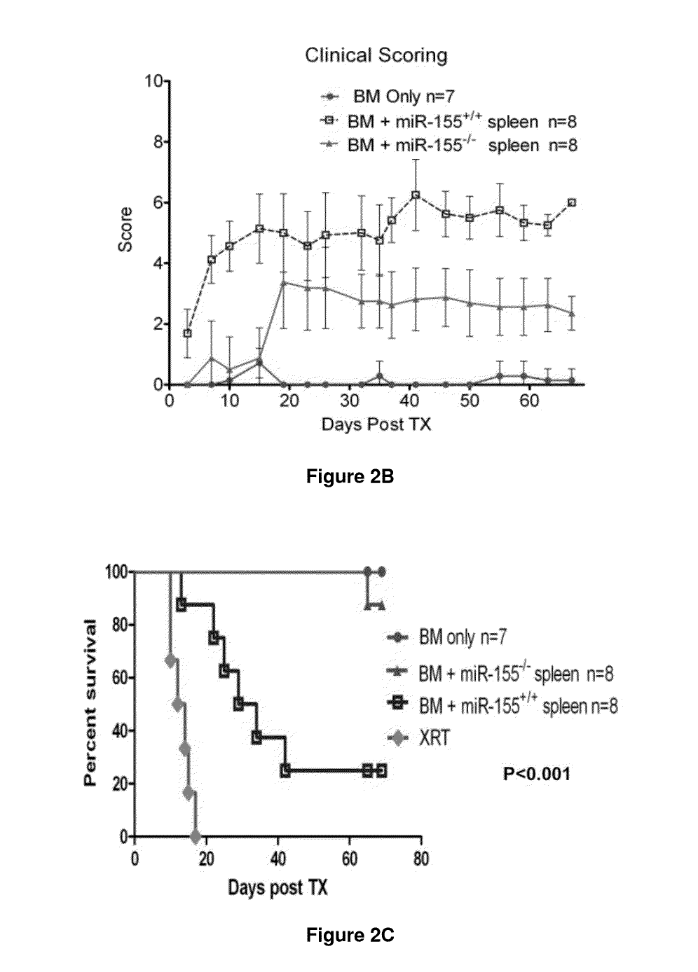

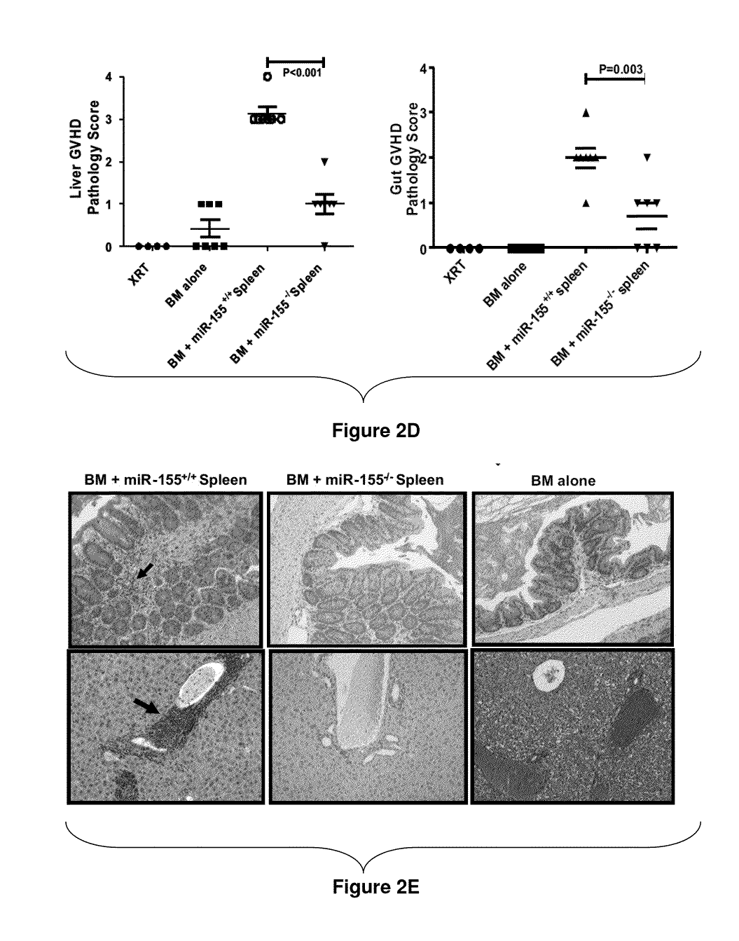

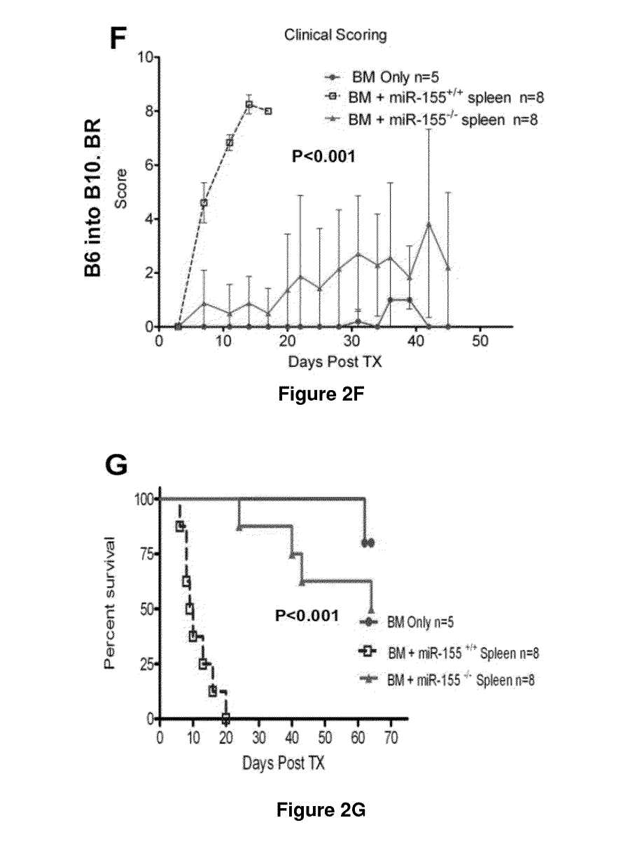

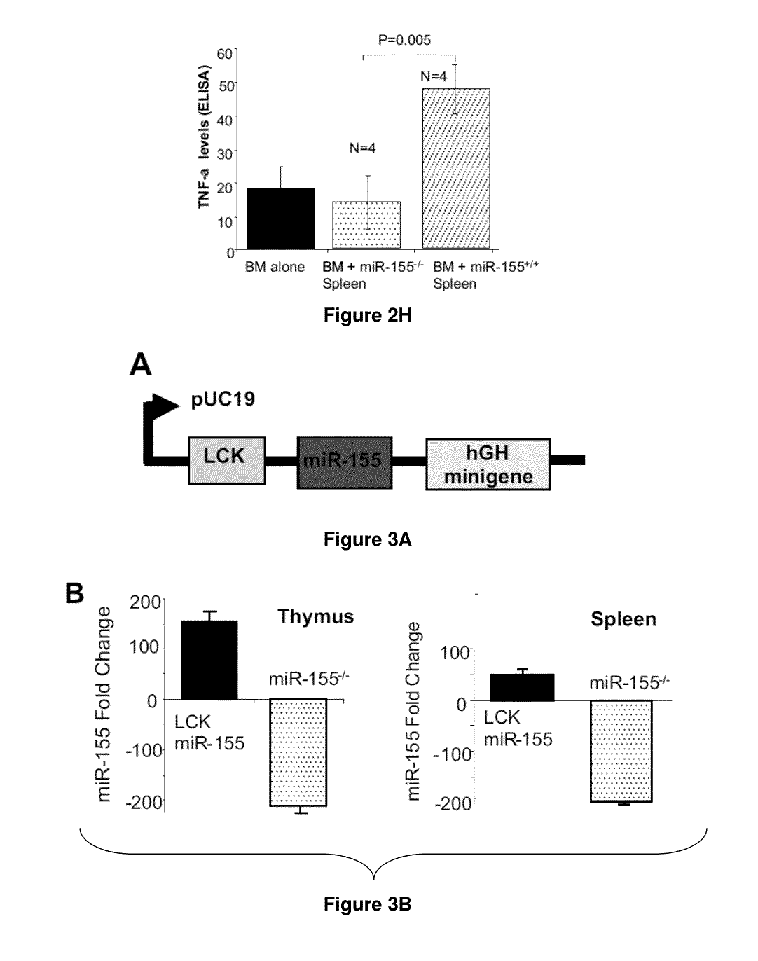

To confirm a causal relationship between miR-155 and aGVHD severity, the MHC mismatched murine experiment was conducted using B6 mice deficient for BIC/miR-155 expression (miR-155^{-/-}) or wild type (WT) miR-155^{+/+} as donors (FIG. 2A).

The miR-155^{-/-} mice exhibit no miR-155 expression in CD4⁺ cells either at baseline or after cross-linking T cell activation (FIG. 9).

Acute GVHD incidence and severity was significantly reduced in recipients of miR-155^{-/-} spleen cells, as evidenced by clinical GVHD scores (t-test P=0.0007, FIG. 2B).

Overall survival was also improved in these mice with respect to WT, (log-rank test P=0.001, FIG. 2C).

There was no difference in CD4⁺ and CD8⁺ T cell numbers between miR-155^{-/-} or wild type (WT) miR-155^{+/+} splenocytes using FACS analysis. Pathological analysis performed in a second cohort of mice confirmed the clinical findings (FIG. 2D).

GVHD scores of the liver and colon obtained on day 15 post-BMT were significantly lower in recipient mice transplanted with miR-155 deficient mice donors, as compared to mice receiving transplants from WT donors (FIG. 2D and FIG. 2E).

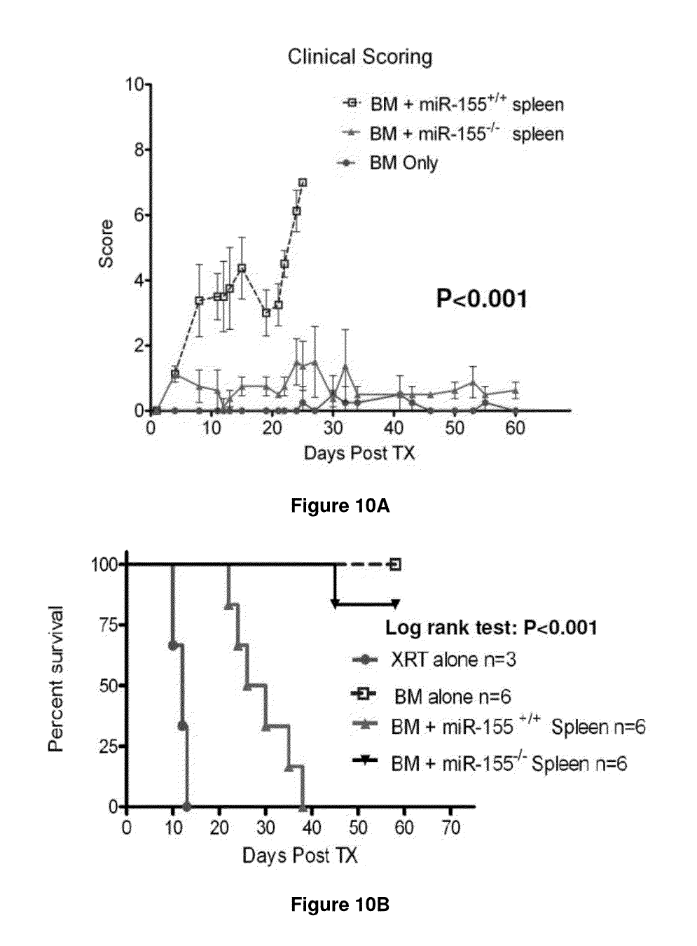

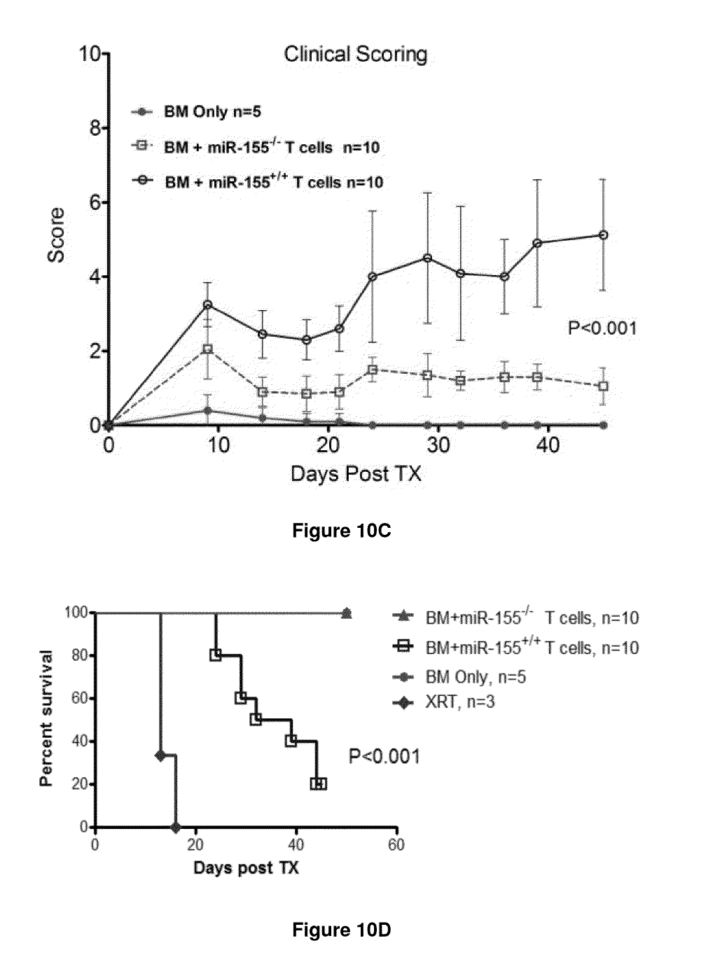

A MHC mismatched GVHD murine experiment was conducted, using a second miR-155 knock out model in a B6 background (miR-155-deficient^{m2/m2}). These mice also do not express miR-155 in T cells and differ from the BIC/miR-155 mice in the strategy used to generate the knock out. Lower aGVHD clinical scores and increased survival were observed for F1 recipients of B6-miR-155-deficient^{m2/m2} cells (FIG. 10A and FIG. 10B).

The lack of severe aGVHD in recipients of miR-155 deficient splenocytes was also independently validated in a different MHC mismatched aGVHD murine model (B6 donors into lethally irradiated B 10.BR(H-2^{k})¹⁹ performed in a different colony environment. Recipients of miR-155-deficient spleen cells had significantly lower clinical GVHD scores and exhibited increased overall survival compared to recipients of WT spleen cells (FIG. 2F and FIG. 2G).

To further identify the miR-155 deficient cell population that protect from aGVHD, we performed the B6 into F1 parental model described above, but using donor untouched T cells (2×10⁶) instead of unfractionated splenocytes. Acute GVHD incidence and severity was significantly reduced in recipients of miR-155^{-/-} T cells, as evidenced by clinical GVHD scores (t-test P<0.001, FIG. 10C). Overall survival was also improved in these mice with respect to WT, (log-rank test P<0.001, FIG. 10D).

Serum TNF-α Expression Levels are Lower in Recipients of miR-155 Deficient Splenocytes.

High levels of soluble TNF-α is characteristic for aGVHD. Also, higher levels promote T cell activation, proliferation and contribute to tissue damage. Murine miR-155 deficient CD4⁺ cells exhibit a Th2 cytokine profile opposite to the Th1 cytokine profile observed during aGVHD. Thus, the inventors measured the expression of TNF-α in the serum of lethally irradiated F1 recipients of BM alone, BM+miR-155^{-/-} splenocytes and BM+miR-155^{+/+} splenocytes (WT), 15 days after transplant by using an ELISA assay. Mice receiving miR-155 deficient spleen cells had significantly lower serum TNF-α level than WT controls (FIG. 2H). Thus, aGVHD reduction in recipients of miR-155^{-/-} vs. miR-155^{+/+} splenocytes is associated with lower sera TNF-α levels.

Recipient Mice of Donor Splenocytes Over-Expressing miR-155 in T Cells Exhibit Rapidly Evolving aGVHD and Short Survival.

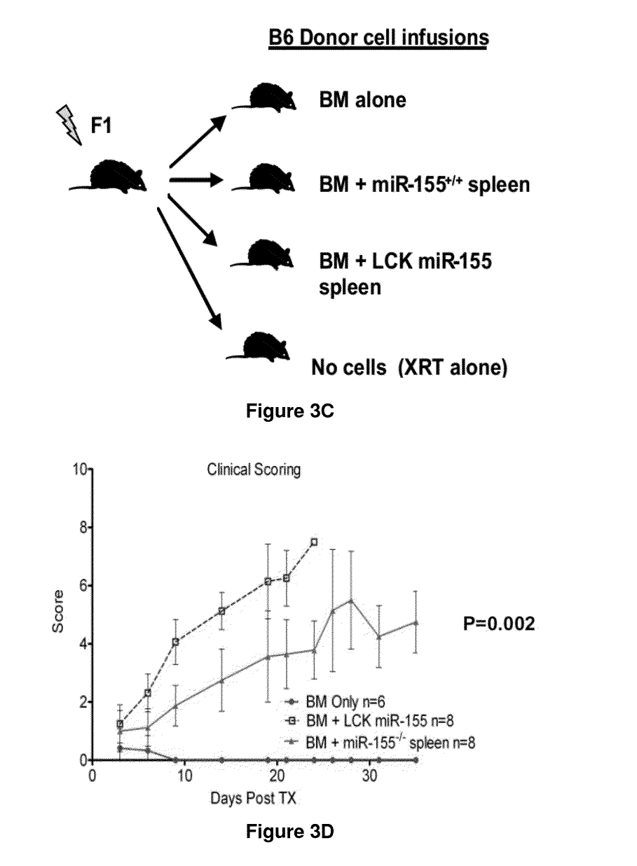

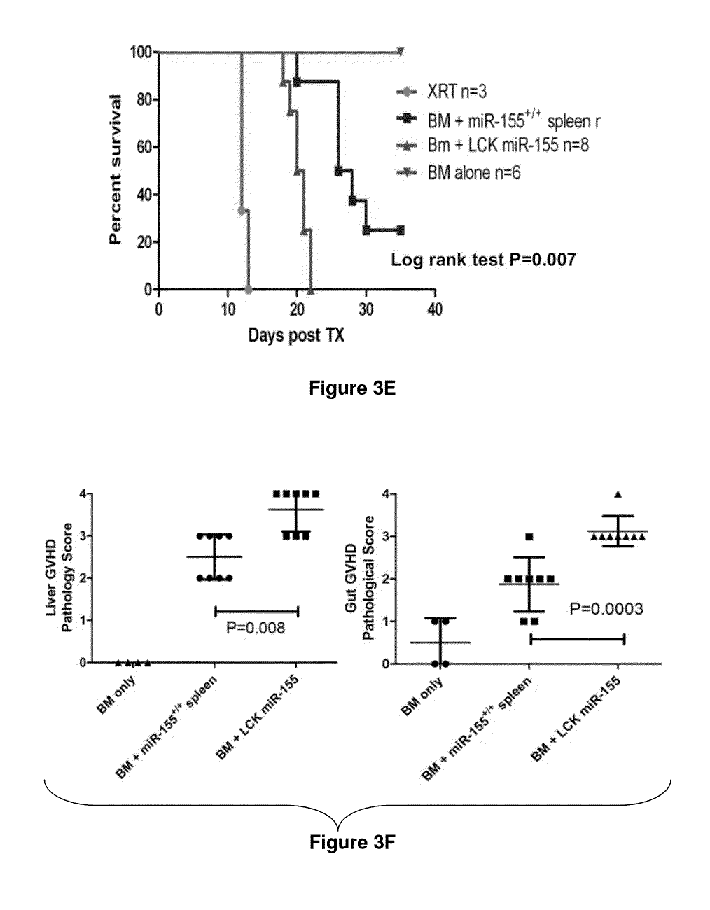

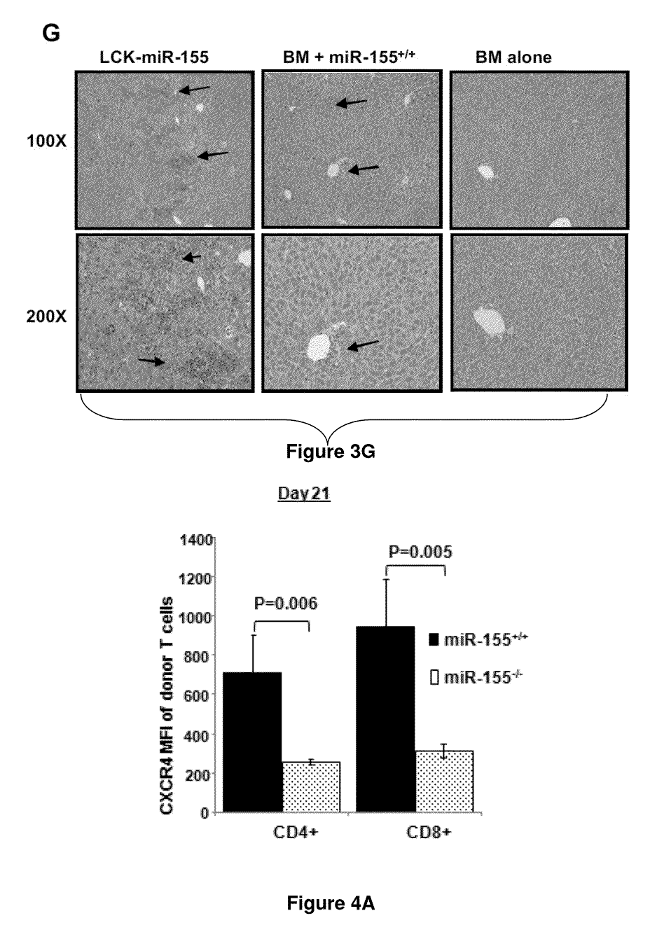

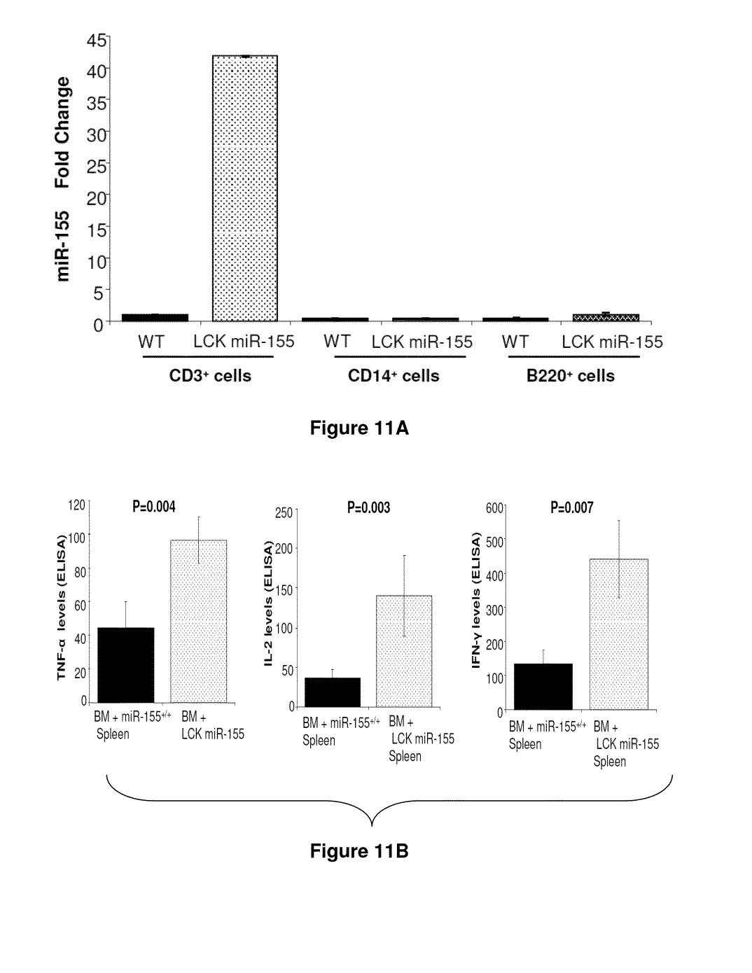

To further establish the regulatory role of miR-155 in aGVHD, a transgenic mouse that over-expresses miR-155 in T cells under the LCK promoter was generated (FIG. 3A-FIG. 3B and FIG. 11A) and performed the MHC mismatched HSCT experiments, but using the LCK-miR-155 transgenic splenocytes as donors (FIG. 3C).

Acute GVHD incidence and severity was significantly increased in recipients of miR-155 transgenic cells with respect to miR-155 WT, as evidenced by clinical GVHD scores (t-test P=0.002, FIG. 3D).

Recipients of miR-155 transgenic cells died earlier than miR-155 WT recipients after transplant (log-rank test P=0.007, FIG. 3E).

GVHD scores of the liver and colon obtained on day 14 post-BMT were significantly higher in recipient mice transplanted with miR-155 over-expressing donor T cells compared to mice receiving transplants from WT donors (t test P=0.008 and P=0.0003, respectively, FIG. 3F and FIG. 3G).

Cytokine measurements revealed higher levels of TNF-α, IL-2 and IFN-γ in recipients of LCK-miR-155 mice with respect to WT recipients (TNF-α, 45±14 vs. 96±13 P=0.004; IL-2, 36±11 vs. 140±50 P=0.003; IFN-γ, 133±40 vs. 440±113 P=0.007, FIG. 11B).

miR-155 does not Affect the all Reactive or Homeostatic Proliferative Potential of T Cells

miR-155 modulates aGVHD in vivo. miR-155 is up-regulated during T cell activation and promotes Th1 differentiation and robust TNF-α secretion. miR-155 up-regulation during aGVHD plays a pivotal role in maintaining a robust Th1 response and effector T cell proliferation and activation during this process.

In contrast, miR-155 deficient T cells have dampened responses to alloantigen stimulation and proliferate less than miR-155 WT counterparts, resulting in reduced aGVHD. To confirm, in vitro experiments were performed where carboxyfluorescein succinimidyl ester (CFSE) labeled CD4+ T cells from miR-155^{-/-} and WT B6 mice were incubated with bone-marrow derived F1 dendritic cells. Donor T cell activation (CD25 and CD69 expression), lymphocyte proliferation and cytokine secretion (IL-2 and TNF-α) were measured over time.

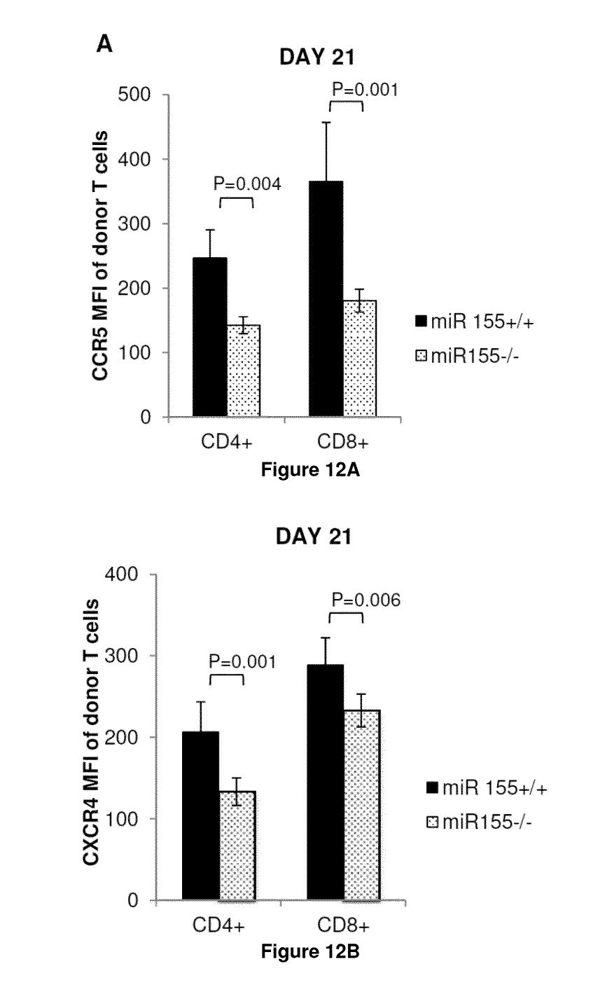

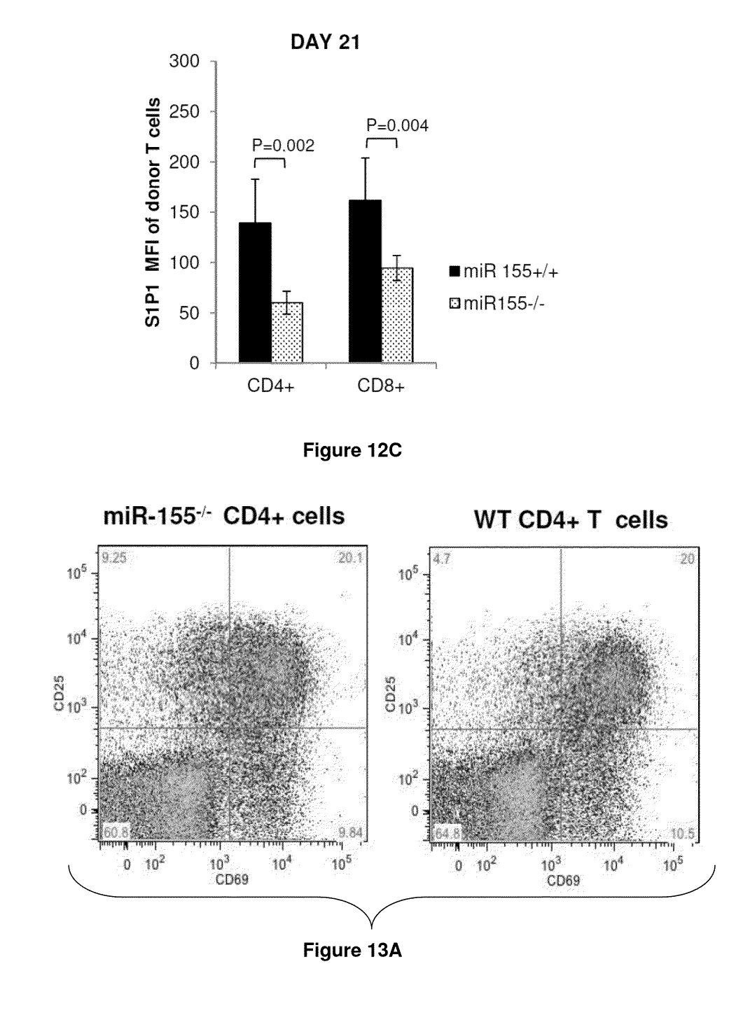

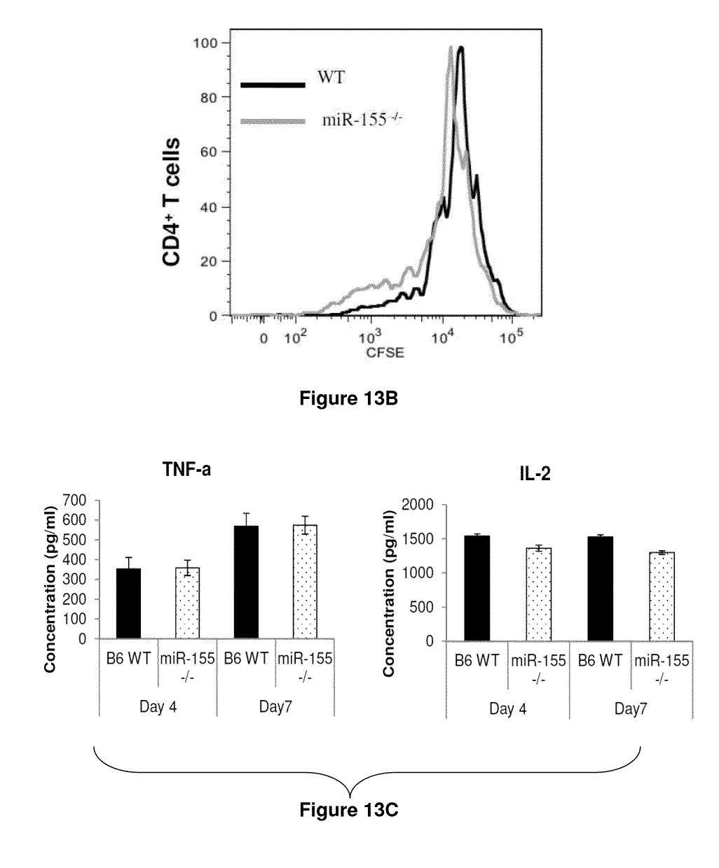

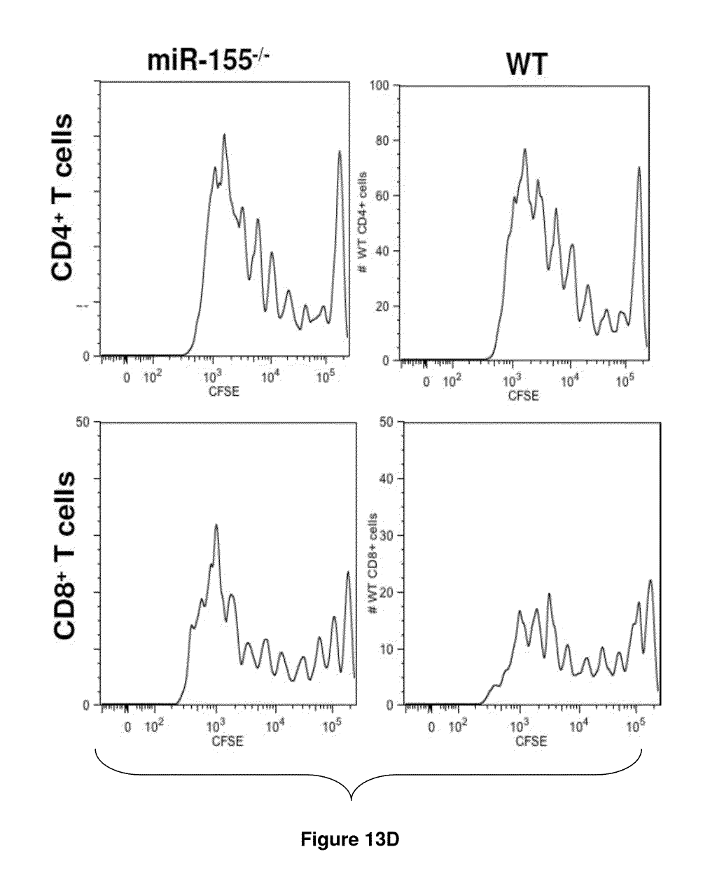

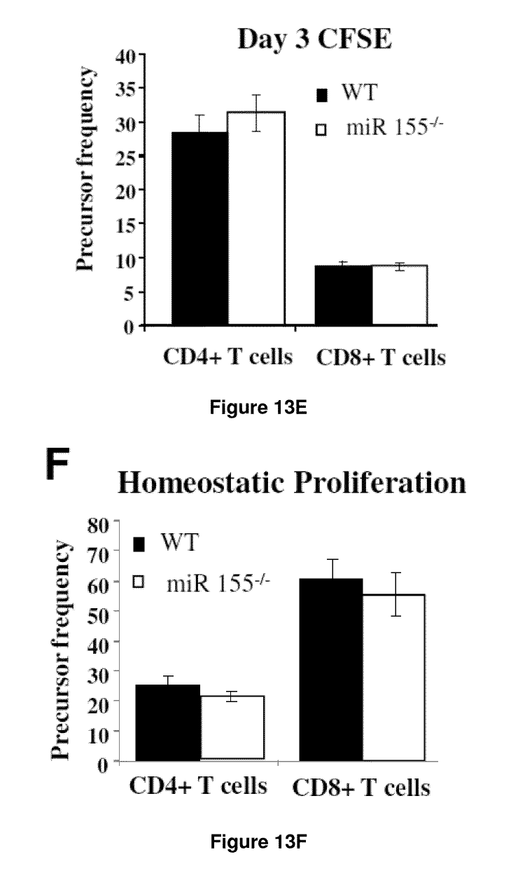

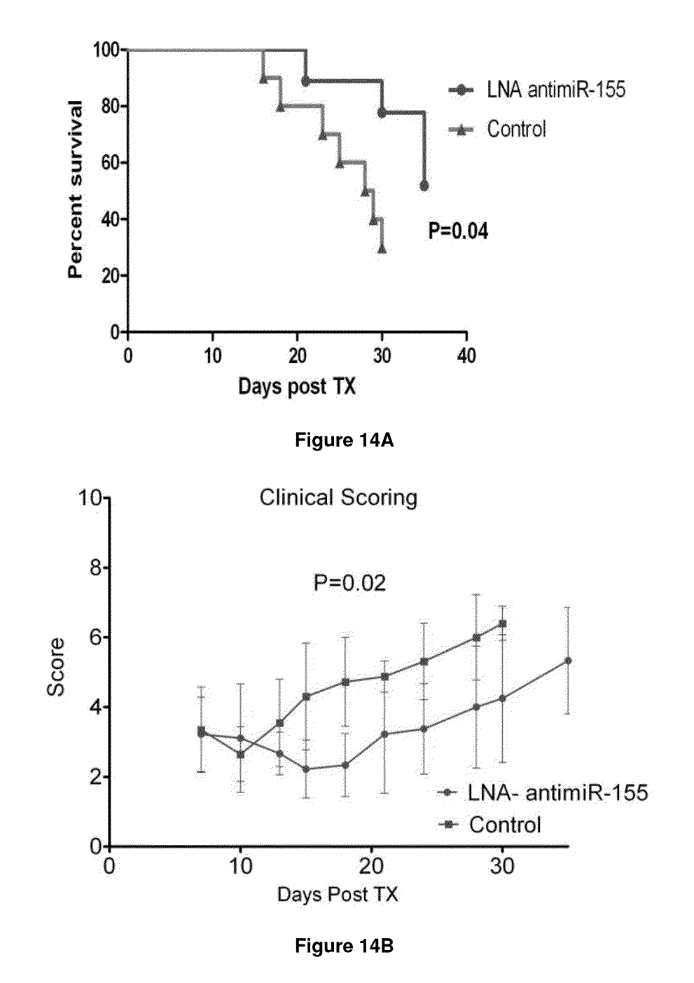

No differences were observed in T cell activation markers, proliferation and cytokine secretion between miR-155^{-/-} and WT B6 splenocytes after alloantigen stimulation in vitro up to 96 hours (FIG. 13A-FIG. 13C). These results were confirmed in vivo. The B6 into F1 MHC mismatched murine experiment was conducted, using T cell depleted BM and CFSE labeled B6 miR-155^{-/-} or WT splenocytes as donors. Spleens were harvested on day 3 post transplant and the precursor frequency of donor T cells was determined. There was no difference between the all reactive proliferative response of the miR-155^{-/-} and miR-155^{+/+} donor T cells in vivo (FIG. 13D and FIG. 12E).

The homeostatic proliferative potential of miR-155^{-/-} T cells was also examined. Congenic lethally irradiated B6 CD45.1 mice received BM cells as well as CFSE labeled CD45.2 miR-155^{+/+} or miR-155^{-/-} splenocytes. Three days later, spleens were harvested and the precursor frequency was determined on the donor CD45.2 T cells. There was no difference in the proliferative response between the WT B6 and miR-155 deficient T cells (FIG. 13F). Thus, these results show that miR-155 does not affect the all reactive or homeostatic proliferative potential of T cells.

miR-155 Modulates Chemokine Receptor Expression of Allogeneic Donor T Cells.

One step in the pathophysiology of aGVHD involves the migration of activated donor T cells to and from secondary lymphoid organs (SLO) to the aGVHD target organs. Two major families of G protein coupled receptors (including the chemokine receptors (CCR7, CXCR4, CCR5) and the sphingosine-1-phosphate (S1P1) receptors), play an important role in orchestrating the migration of leukocytes to and from the SLOs. The inventors determined whether alteration in donor T cell trafficking patterns is one of the mechanisms via which miR-155 modulates aGVHD.

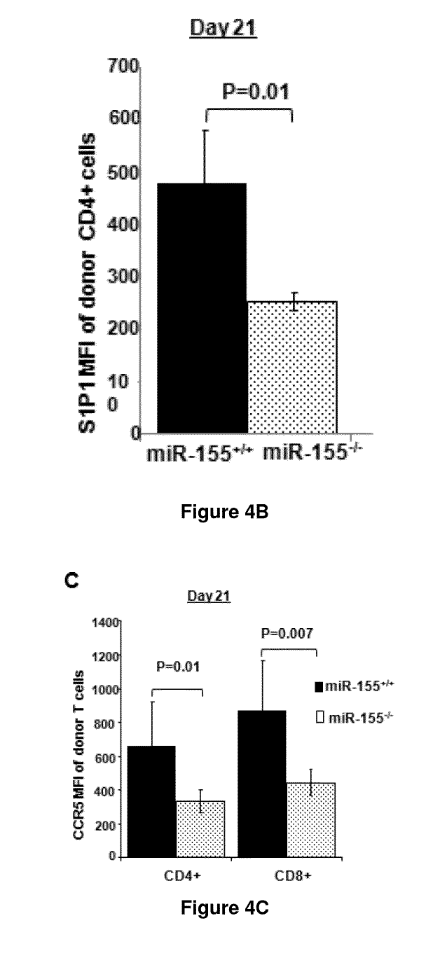

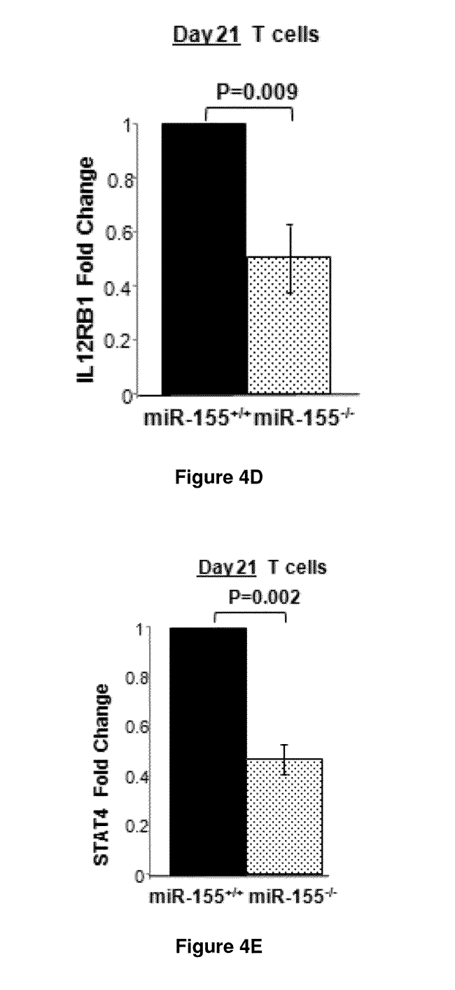

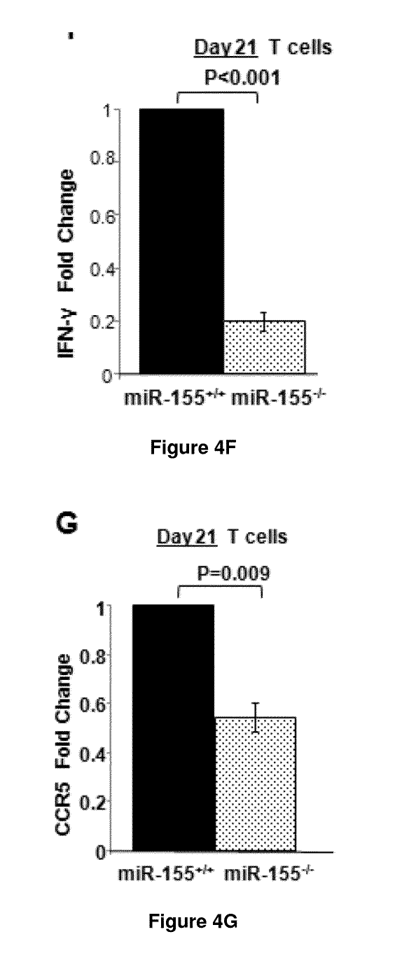

The expression levels of the major homing receptors CCR5, CXCR4, CCR7 and S1P1 on donor T cells isolated from recipient splenocytes on early (day 4) and late (day 21) time points post-transplant using flow cytometry was determined. No significant differences in the expression pattern of CCR7 between T cells of the miR-155 deficient and B6 WT cells at the time points investigated were found. While there were no differences in expression at the early time point, CXCR4 was significantly down-regulated on miR-155 deficient CD4⁺ (251.66±14.01 vs. 711±188.83, P=0.006) and CD8⁺ (310.33±38.73 vs. 940.66±243.79, P=0.005) T cells, as compared to WT B6 T cells at day 21 (FIG. 4A).

There was a significant decrease in the surface expression of S1P1 on splenic CD4⁺ miR-155 deficient T cells, as compared to WT B6 (253.33±17.03 vs. 476±101.79, P=0.01) at day 21, while no differences were observed at the earlier time point (FIG. 4B).

With respect to CCR5, its expression was significantly lower on CD4⁺ and CD8⁺ miR-155 deficient donor T cells compared to WT B6 (CD4⁺; 381.66±38.03 vs. 787.33±268.06, P=0.01 and CD8⁺: 495±40.95 vs. 969.66±369.15, P=0.007) at day 21 (FIG. 4C).

Complementary to the above findings, reduced donor lymphocyte infiltration in aGVHD target organs of mice that received miR-155 deficient splenocytes as compared to those that received WT B6 splenocytes was noted (FIG. 2D and FIG. 2E).

To determine how miR-155 regulates CCR5 (the primary chemokine receptor that orchestrates migration of donor T cells to recipient peripheral target organs such as liver), T cells were isolated from recipient mice on day 21, and mRNA expression levels of candidate genes known to regulate CCR5 by RT-PCR were measured.

The IL-12 signaling pathway regulates CCR5 expression on activated T cells, and IL-12R expression on T cells is negatively regulated by SOCS-1, a validated direct target of miR-155 in T cells. As shown in FIG. 4D, IL-12RB1 expression levels were downregulated in miR-155^{-/-donor} T cells with respect to controls. In addition, STAT-4, which is a major biological mediator of the downstream signaling events initiated by IL-12R activation in lymphocytes and is needed for the induction of CCR5 on T cells, was found downregulated in miR-155^{-/-} T cells (FIG. 4E).

Likewise, IFN-γ, which is another important IL-12 and STAT-4 regulated gene, was downregulated in miR-155 deficient cells (FIG. 4F).

These results show that CCR5 mRNA expression levels were down-regulated as well in miR-155 deficient T cells (FIG. 4G).

These results also show a significant decrease in the expression of homing receptors CCR5, CXCR4 and S1P1 receptors on miR-155 deficient donor T cells. The down-regulation of these homing receptors in donor T cells thus impairs the migration of these cells to the peripheral targets organs resulting in less aGVHD.

Blocking miR-155 Expression Using a Seed-Targeting LNA-antimiR-155 Oligonucleotides Decreases the Severity of aGVHD and Prolonged Survival after HSCT in Recipient Mice.

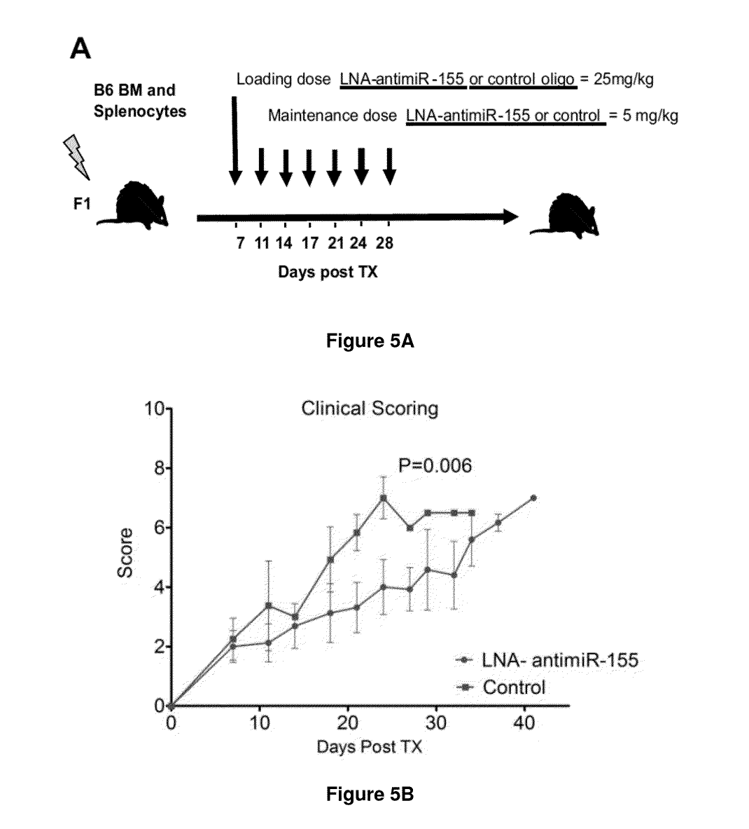

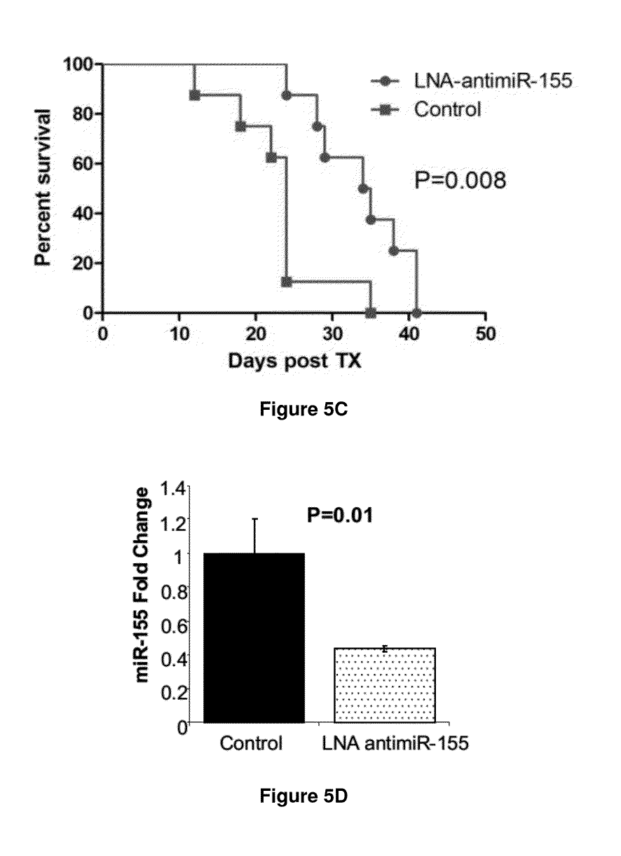

Blocking miR-155 expression using LNA-antimiR-155 prevents and/or delays the development of aGVHD disease after HSCT. The inhibition of miRNA function in cultured cells and in vivo can be achieved using fully LNA-modified 8-mer antimiR oligonucleotides complementary to the miRNA seed region. Lethally irradiated recipient F1 mice were treated with LNA-antimiR-155 (n=10) or an 8-mer LNA control oligonucleotide (n=10) (loading dose 25 mg/kg and 5 mg/Kg I.V. twice weekly) starting at day 7 up to day 30 after infusion of donor B6 splenocytes (FIG. 5A).

Treatment with antimiR-155 decreased the severity of clinical aGVHD (FIG. 5B) and prolonged the survival of recipient mice post transplant (FIG. 5C).

Down-regulation of miR-155 was observed in the spleen of the mice treated with antimiR-155 with respect to the controls (FIG. 5D).

miR-155 Deficiency in Donor T Cells does not Abrogate Graft-Versus Leukemia Effects.

aGVHD and graft-versus-leukemia effect (GVL) are two highly linked immune reactions. To address the effects of miR-155 on GVL early after transplantation, unfractionated splenocytes (20×10⁶) from either miR-155^{-/-} B6 (B6,H-2^{b}) or WT B6 mice donors along with T cell depleted BM (5×10⁶) cells from WT B6 mice were transplanted into lethally irradiated F1 mice. In addition, a luciferase transduced murine leukemic cell line P815 (1×10⁴) was transplanted along with the splenocytes. Separate cohorts were used for imaging and survival.

As shown in FIG. 6A and FIG. 6B, there were no significant differences in the leukemic burden in recipient tissues as measured by bioluminescence assays and histopathology between miR-155^{-/-} vs. WT B6 splenocytes recipients, thus showing retention of GVL effect in miR-155^{-/-} donor T cells.

Recipients of miR-155^{-/-} spleen cells+P815 (n=8) survived longer than WT spleen cells recipients+P815 (n=8) (Log rank test P=0.01) (FIG. 6C). Two mice from the miR-155^{-/-} cohort died from leukemia (as defined by the presence of hind limb paralysis), though like the remaining six mice from that group there was no significant evidence of leukemic infiltration in the spleen or liver by histopathology, as shown in a representative case (FIG. 6B). The cohort of mice who received miR-155^{+/+} WT spleen cells died earlier from aGVHD and in one case from leukemia defined only by hind limb paralysis. The BM only cohort died prematurely from disseminated leukemia (FIG. 6B and FIG. 6C).

miR-155 Expression is Up-Regulated in the Intestinal Tract from Patients with aGVHD.

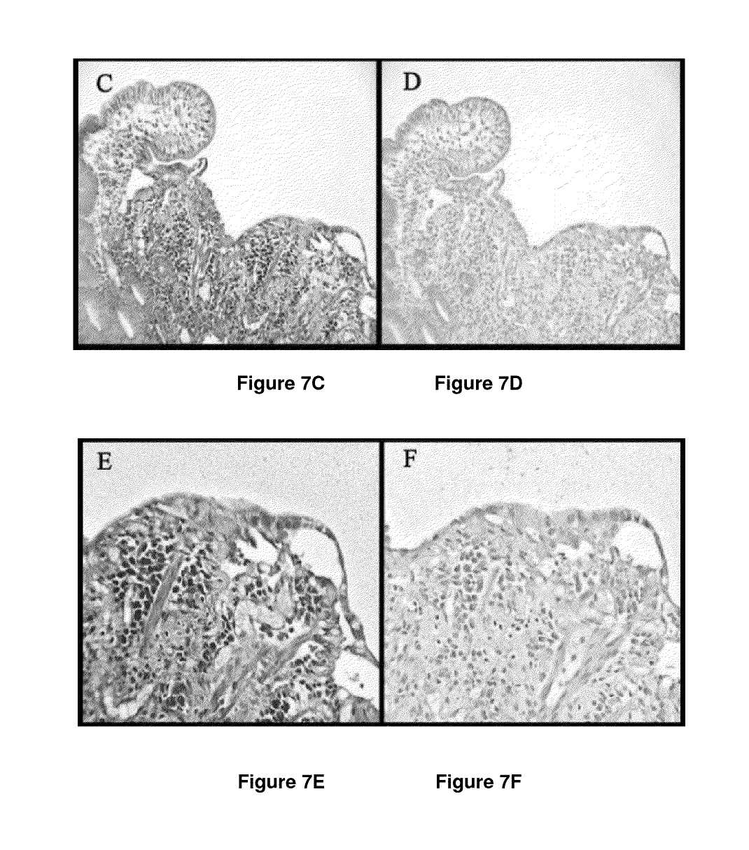

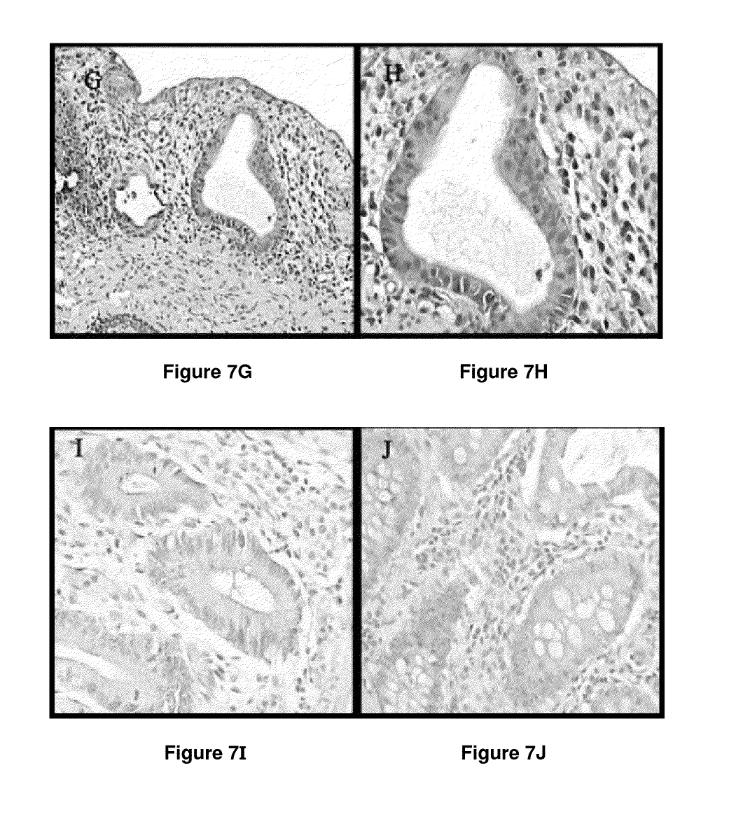

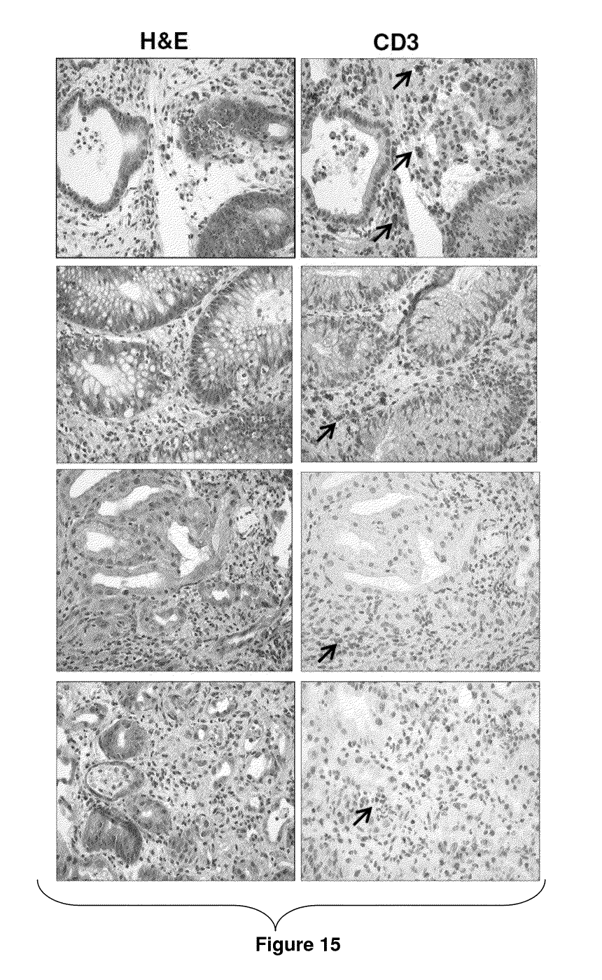

miR-155 expression was measured using LNA-based in situ hybridization from clinically and histologically confirmed small and large bowel biopsies of aGVHD patients (n=5), and healthy controls (n=3) (FIGS. 7A-7J). A strong up-regulation of miR-155 expression in the inflammatory cells from all patients with small and large bowel aGVHD was found, while miR-155 expression was absent in normal bowel. Further immunohistochemistry studies confirmed that the inflammatory cells were T lymphocytes (FIG. 15). Thus, these results show that high levels of miR-155 are relevant in human aGVHD.

Materials and Methods

Cytokine ELISA Measurements

Cytokines levels (TNF-α, IL-2, IFN-γ) in plasma and cell supernatants were measured by 2-site sandwich ELISA. Samples were assayed in triplicate and results are shown as the means from at least three pooled biological samples (each one in triplicate) within a group.

Purification of T-Cell Subsets

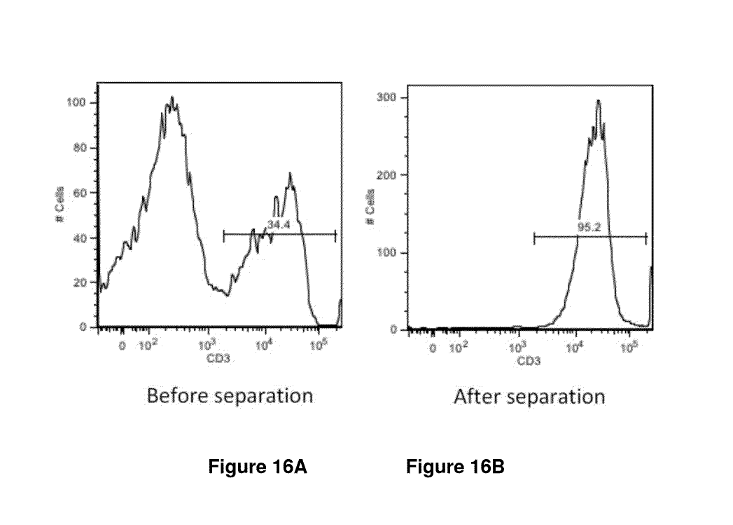

To isolate activated CD8⁺ T cells from the spleen, splenocytes were stained with a Phycoerythrin conjugated (PE)-anti CD8 and a FITC-antiCD44 antibodies and sorted by CD8⁺ CD44⁺ CD8⁺ CD44⁻ T cells by using fluorescence activated cell sorting (FACS) ARIA. The purity of CD8⁺ T cells was >90%. T cell depleted bone marrow samples were obtained from mice using anti-CD3episilon-biotin antibody conjugated with magnetic microbeads followed by magnetic cell sorting (miniMACS) as per manufacturer's protocols. An aliquot of bone marrow cells was taken before and after T cell depletion for flow cytometry to confirm depletion. Donor untouched CD4⁺ cells were purified from spleens using negative selection CD4⁺ isolation kit per manufacturer's recommendations (miniMACS). To further separate CD4⁺ CD62L high and CD4⁺ CD62L low, untouched CD4⁺ cells obtained by negative selection were incubated with anti-CD62L conjugated magnetic microbeads and followed by magnetic cell sorting. Eluates containing CD4⁺ CD62L high and flow through CD4⁺ CD62L low were used for experiments. To isolate untouched T cells from B6 miR-155 deficient or WT mice spleens we used the mouse Pan T cell isolation kit IL The kit is based on a cocktail of biotin-conjugated antibodies against CD11b, CD11c, CD19, CD45R (B220), CD49b (DX5), CD105, Anti-MHC-class II, and Ter-119. This ensures the elimination of red blood cells (Ter-119) donor antigen presenting cells (CD11b, CD11c, anti-MHC-class II), B cells (CD19, CD45R) and NK cells (CD49b). The purity of the isolated T cells was greater than 95% by FACS using CD3 as the marker as in FIG. 16A-FIG. 16B.

In Vitro T Cell Activation Assays

Isolated CD4⁺ cells (2×10⁶) from B6 miR-155 deficient or WT murine splenocytes were cultured in 6-well plates with RPMI 1640, supplemented with L-glutamine (2 nM), penicillin (100 U/ml), streptomycin (0.1 mg/ml), 2-mercaptoethanol (5×10⁻⁵ M), 10% fetal calf serum coated with 2 ug/ml or 5 ug/ml each of αCD3 (clone 145-2C11; BD Biosciences) and soluble CD28 (clone 37.51; BD Biosciences). After incubation for 4, 12, 24 and 48 hours the supernatant was collected for cytokine measurements and cell pellets were placed on Trizol.

In Vitro T Cell Proliferation Assay

CD4+ T cells were isolated from B6 miR-155 deficient or WT murine splenocytes and cultured with F1 (allogeneic) or B6 (syngeneic, negative control) bone-marrow derived dendritic cells (stimulator: effector=1:5). At the end of 4 days, cells were analyzed for CD25 and CD69 surface expression as a marker for activation using a LSRII (Becton Dickinson, Mountain View, Calif.) and FACSDiva software (Becton Dickinson). At the end of 4 and 7 days of culture, supernatant was collected for cytokine measurements by ELISA. For proliferation, the CD4+ cells were stained with CFSE prior to incubation with F1 (allogeneic) or B6 (syngeneic, negative control) bone-marrow derived dendritic cells and CFSE divisions were measured after 7 days using LSRII (Becton Dickinson, Mountain View, Calif.) and FACSDiva software (Becton Dickinson).

In Vivo Proliferation Assay

B6 miR-155^{-/-} or miR155^{+/+} splenocytes were stained with 5 μM CFSE (Molecular Probes, Eugene, Oreg.) at a concentration of 10×10⁶/mL in PBS at room temperature for 10 minutes with gentle shaking. Labeling was quenched by the addition of the 5 volumes of ice-cold media (RPMI with 10% FCS) and kept on ice for 10 minutes followed by washing 2 times with cold media. CFSE-stained B6 splenocytes (20×10⁶) and t-BM (5×10⁶) were infused into lethally irradiated F1 mice. Spleens were harvested 3 days after transplantation. CFSE divisions were evaluated on gated donor (H2 Kd negative) CD4⁺ and CD8⁺ cells using a LSRII (Becton Dickinson, Mountain View, Calif.) and FACSDiva software (Becton Dickinson). Calculations for precursor frequency and proliferative capacity were performed.

Nanostring nCounter microRNA Experiments and Data Analysis

The NanoString nCounter Human miRNA Expression Assay Kit was used to profile more than 700 human and human-associated viral miRNAs in donor T cells from recipient mice with aGVHD or controls. 100 ng of total RNA was used as input for nCounter miRNA sample preparation reactions. All sample preparation was performed according to manufacturer's instructions (NanoString Technologies). Preparation of small RNA samples involves the ligation of a specific DNA tag onto the 3′ end of each mature miRNA. These tags are designed to normalize the Tm's of the miRNAs as well as to provide a unique identification for each miRNA species in the sample. The tagging is accomplished in a multiplexed ligation reaction using reverse-complementary bridge oligonucleotides to direct the ligation of each miRNA to its designated tag. Following the ligation reaction, excess tags and bridges are removed, and the resulting material is hybridized with a panel of miRNA:tag-specific nCounter capture and barcoded reporter probes. Hybridization reactions were performed according to manufacturer's instructions with 5 μl of the 5-fold diluted sample preparation reaction. All hybridization reactions were incubated at 64° C. for a minimum of 18 h. Hybridized probes were purified using the nCounter Prep Station (NanoString Technologies) following the manufacturer's instructions to remove excess capture and reporter probes and to immobilize transcript-specific ternary complexes on a streptavidin-coated cartridge. Data collection was carried out on the nCounter Digital Analyzer (NanoString Technologies) following the manufacturer's instructions to count individual fluorescent barcodes and quantify target RNA molecules present in each sample. For each assay, a high density scan (600 fields of view) was performed. The samples analyzed using the Nanostring technology are subject to lane-to-lane variations that can arise during processing. To minimize the impact of these sample preparation and detection anomalies, the data were normalized to the positive control count values. The positive controls are synthetic miRNA sequences included in the assay to confirm successful ligation of the miRNA to the tags. This type of normalization is called “Technical Normalization.” Next, a biological normalization method “Quantile Normalization” has been applied. To identify differentially expressed miRNAs between samples from two classes we used the non-parametric statistical test called Significance Analysis of Microarrays (SAM). SAM identifies miRNAs with statistically significant changes in expression by assimilating a set of miRNA-specific t tests. Once the set of significant (Positive and Negative) miRNAs have been computed, a cluster analysis has been conducted to identify subgroups of significant miRNAs sharing a similar expression level. This has been done through the Average Linkage hierarchical clustering algorithm. These results have been obtained using the SAM and cluster analysis module of the Tmev system.

Locked Nucleic Acid (LNA) In Situ Hybridization.

Locked nucleic acid (LNA) modified probes complementary to human mature miR-155 (5′ACCCCUAUCACGAUUAGCAUUAA-3′) [SEQ ID NO:1] and scrambled negative control (5′-UUCACAAUGCGUUAUCGGAUGU-3′) [SEQ ID NO:2] digoxigenin-labeled at the 5′ position were purchased from Exiqon (Vedbaek, Denmark). Detection of mature miRNAs by in situ hybridization utilizing oligonucleotide probes was performed. Briefly, frozen control and aGVHD colon specimens were formalin-fixed, embedded in wax and subsequently treated with proteinase K (Ventana Medical Systems Protease 1 in RNase-free water for 4 minutes. After co-denaturation at 60° C. for 5 min, hybridization was performed at 37° C. overnight followed by a low stringency wash in 0.2×SSC and 2% bovine serum albumin at 4° C. for 10 min. The probe-target complex was visualized utilizing a digoxigenin antibody conjugated to alkaline phosphatase acting on the chromogen nitro blue tetrazolium/5-bromo-4-chloro-3-indolyl phosphate. Therefore, positive expressing miRNA cells stain dark blue. Nuclear fast red served as the counterstain. Identical control experiments were performed utilizing a digoxigenin-labeled scrambled miRNA and control experiments were conducted where the miRNA probe was omitted.

Immunohistochemistry

Briefly, formalin-fixed paraffin embedded sections of human small and large bowel samples were examined by hematoxylin and eosin stain, and CD3 immunostains performed using mouse monoclonal anti-human CD3 Ab (DAKO) and visualized using DAKO LSAB+/HRP kit.

Discussion

miR-155 regulates the severity of aGVHD after allogeneic HSCT by a combination of miR-155 dependent effects on T cell homing and TNF-α secretion. There is a significant downregulation of CCR5 mRNA and protein expression in miR-155 deficient donor T cells after engraftment (day 21) which is now shown herein to be caused by reduced IL-12R levels and dampened downstream signaling pathways via STAT-4.

CCR5 plays an important role in directing the migration of donor T cells towards the liver. The downregulation of CCR5 on the donor miR155^{-/-} T cells contributes to the dramatic decrease of donor lymphocytic infiltration in the recipient live, as confirmed by histology.

In addition, there is a marked decrease in the expression of SIP1 in donor miR-155 deficient T cells after HSCT. (SIP1 downregulation via administration of FTY720 leads to sequestration of T cells within secondary lymphoid organs, decreasing lymphocyte migration and tissue infiltration).

There are decreased levels of CXCR4 in donor miR-155 deficient T cells at day 21 after HSCT. CXCL12/SDF-1 and its receptor CXCR4 have a major role in directing the migration of progenitors during hematopoiesis and a minor role in T cell activation, proliferation and migration to peripheral lymph nodes. CXCR4 downregulation in miR-155 deficient T cells contributes to a reduction in the inflammatory function/phenotype and migration of these cells. Overall, the combined effects of CXCR4, CCR5 and S1P1 down-regulation in donor T cells show that alterations in cell trafficking and homing are one of the mechanisms by which miR-155 modulates the severity of aGVHD in mice.

The examples herein show that miR-155 up-regulation during aGVHD maintains the high levels of TNF-α and perpetuates inflammation during this process, in particular regarding to gut aGVHD.

Blocking miR-155 expression in donor T cells after HSCT resulted in less aGVHD and improved survival. Also, the loss of miR-155 expression did not abrogate GVL effects. Many hematological malignancies over-expresses miR-155 and allogeneic HSCT is used frequently as consolidation therapy for these diseases. Suppressing miR-155 expression provides benefits, only for aGVHD prevention but also for minimal residual disease control.

miR-155 expression is up-regulated in T cells from mice with aGVHD. Modulation of miR-155 expression on these cells dramatically affects aGVHD incidence and severity in a murine model of aGVHD.

Also, blocking miR-155 expression in vivo by LNA-antimiR-155 after allogeneic HSCT prevents lethal aGVHD in mice.

Up-regulation of miR-155 was found in clinical specimens from patients with intestinal aGVHD. Modulation of the expression of miR-155 is a useful target for therapeutic intervention in aGVHD.

### Example

### Therapeutic/Prophylactic Methods and Compositions

Various delivery systems are known and are used to administer a therapeutic of embodiments of the invention, e.g., encapsulation in liposomes, microparticles, microcapsules, expression by recombinant cells, receptor-mediated endocytosis, construction of a therapeutic nucleic acid as part of a retroviral or other vector, etc. Methods of introduction include, but are not limited to, intradermal, intramuscular, intraperitoneal, intravenous, subcutaneous, intranasal, and oral routes. The compounds are administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of embodiments of the invention into the central nervous system by any suitable route, including intraventricular and intrathecal injection; intraventricular injection may be facilitated by an intraventricular catheter, for example, attached to a reservoir, such as an Ommaya reservoir.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, the implant being of a porous, non-porous, or gelatinous material, including membranes, such as sialastic membranes, or fibers. In one embodiment, administration is by direct injection at the site (or former site) of a malignant tumor or neoplastic or pre-neoplastic tissue.

In a specific embodiment where the therapeutic is a nucleic acid encoding a protein therapeutic the nucleic acid is administered in vivo to promote expression of its encoded protein, by constructing it as part of an appropriate nucleic acid expression vector and administering it so that it becomes intracellular, or coating with lipids or cell-surface receptors or transfecting agents, or by administering it in linkage to a homeobox-like peptide which is known to enter the nucleus. Alternatively, a nucleic acid therapeutic can be introduced intracellularly and incorporated within host cell DNA for expression, by homologous recombination.

The present invention also provides pharmaceutical compositions. Such compositions comprise a therapeutically effective amount of a therapeutic, and a pharmaceutically acceptable carrier or excipient. Such a carrier includes, but is not limited to, saline, buffered saline, dextrose, water, glycerol, ethanol, and combinations thereof. The carrier and composition can be sterile. The formulation will suit the mode of administration.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition also includes a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it is be dispensed with an infusion bottle containing sterile pharmaceutical grade water or saline. Where the composition is administered by injection, an ampoule of sterile water for injection or saline is provided so that the ingredients are mixed prior to administration.

The therapeutics of embodiments of the invention are formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the therapeutic of embodiments of the invention which will be effective in the treatment of a particular disorder or condition will depend on the nature of the disorder or condition, and is determined by standard clinical techniques. In addition, in vitro assays may optionally be employed to help identify optimal dosage ranges. The precise dose to be employed in the formulation will also depend on the route of administration, and the seriousness of the disease or disorder, and is decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems

The invention also provides a pharmaceutical pack or kit comprising one or more containers filled with one or more of the ingredients of the pharmaceutical compositions of the invention. Optionally associated with such container(s) is a notice in the form prescribed by a governmental agency regulating the manufacture, use or sale of pharmaceuticals or biological products, which notice reflects approval by the agency of manufacture, use or sale for human administration.

In view of the many possible embodiments to which the principles of the inventors' invention may be applied, it should be recognized that the illustrated embodiments are only preferred examples of the invention and should not be taken as a limitation on the scope of the invention. Rather, the scope of the invention is defined by the following claims. The inventors therefore claim as the inventors' invention all that comes within the scope and spirit of these claims.

Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents

What is claimed is:

1. A method for treating or preventing acute graft-versus-host disease (aGVHD) in a subject, comprising:
administering to the subject a miR-155 specific inhibitor in an amount effective to decrease expression of miR-155 in the subject.

2. A method for treating acute graft-versus-host disease (aGVHD) in a recipient of an organ or tissue transplant, comprising:
administering to the transplant recipient a miR-155 specific inhibitor in a pharmaceutically effective amount after the transplantation.

3. A method for decreasing a T-cell activity in an individual suffering from acute graft-versus-host disease (aGVHD), comprising:
administering to the individual an effective amount of a miR-155 specific inhibitor, and a pharmaceutically acceptable carrier; wherein the miR-155 specific inhibitor decreases a T-cell activity in vivo.

4. A method of inhibiting miR-155 expression in a subject having, or at risk of having acute graft-versus-host disease (aGVHD), comprising:
introducing an effective amount of at least one miR-155-specific inhibitor to the subject.

5. A method of treating acute graft-versus-host disease (aGVHD) in a subject, comprising:
administering to the subject a therapeutically effective amount of a miR-155 inhibitor to treat or ameliorate one or more symptoms of aGVHD.

6. A method of treating tissue or organ transplant rejection in a recipient, comprising:
administering to the recipient an effective amount of a miR-155 specific inhibitor composition to attenuate the tissue or organ transplant rejection.

7. A method of modulating the immune response against an organ or tissue transplant in a recipient, comprising:
administering to the recipient an effective amount of a miR-155 specific inhibitor composition to modulate the response against the organ or tissue transplant.

8. A method of treating acute graft-versus-host disease (aGVHD) in a transplant recipient, comprising:
administering to the recipient an effective amount of a miR-155 specific inhibitor composition to attenuate the graft-versus-host-disease in the recipient.

9. The method of claim 1, wherein the miR-155 specific inhibitor partially suppresses TNF-α expression.

10. The method of claim 1, wherein the at least one miR-155-specific inhibitor is selected from the group consisting of pre-miR-155, pri-miR-155, mature miR-155, and seed miR-155.

11. The method of claim 1, wherein the miR-155 specific inhibitor is selected from the group consisting of: anti-miRs and target mimics.

12. The method of claim 1, wherein the miR-155 specific inhibitor is an anti-miR-155 antisense oligonucleotide.

13. The method of claim 12, wherein the antisense oligonucleotide is an antagomir.

14. The method of claim 12, wherein the antisense oligonucleotide is complementary to at least 10 contiguous nucleotides in miR-155.

15. The method of claim 12, wherein the antisense oligonucleotide comprises between 7 and 25 nucleotides.

16. The method of claim 12, wherein the antisense oligonucleotide comprises between 7 and 21 nucleotides.

17. The method of claim 1, wherein the miR-155 specific inhibitor comprises a nucleotide sequence of least 6 consecutive nucleotides that are complementary to positions 2-8 of a seed region of LNA-miR-155 specific inhibitor, and wherein the miR-155 specific inhibitor reduces levels of TNF-α.

18. The method of claim 1, wherein the miR-155 specific inhibitor is chemically modified on at least one nucleotide.

19. The method of claim 18, wherein the chemical modification comprises a locked nucleic acid (LNA).

20. The method of claim 12, wherein one or more of the nucleotide units of the antisense oligonucleotide are locked nucleic acid (LNA) units or 2′ substituted nucleotide analogues.

21. The method of claim 12, wherein one or more of internucleoside linkages between nucleotide units of the antisense oligonucleotide are phosphorothioate internucleoside linkages.

22. The method of claim 18, wherein the chemical modification comprises 2′-O-methyl.

23. The method of claim 8, wherein the miR-155 specific inhibitor comprises a polynucleic acid molecule that is essentially complementary to miR-155.

24. The method of claim 1, wherein the miR-155 specific inhibitor comprises a polynucleic acid molecule that is 100% complementary to miR-155.

25. The method of claim 1, wherein the miR-155 specific inhibitor is complementary to a sequence at least 80% identical to human mature miR-155.

26. The method of claim 1, wherein the miR-155 specific inhibitor is complementary to a sequence at least 80% identical to pre-miR-155.

27. The method of claim 1, wherein the miR-155 specific inhibitor is perfectly complementary to a human miR-155 seed sequence.

28. The method of claim 1, wherein the subject transplant recipient has aGVHD.

29. The method of claim 8, wherein the transplant recipient is at risk of developing aGVHD.

30. The method of claim 8, wherein the transplant recipient has received hematopoietic stem cell transplant or bone marrow transplant.

31. The method of claim 8, wherein the transplant recipient has received hematopoietic stem cell transplant or bone marrow transplant and manifests grade 2 or greater aGVHD.

32. The method of claim 1, wherein the miR-155 specific inhibitor is administered to the subject by at least one administration route selected from the group consisting of: oral, parenteral, intravenous infusion, and injection.

33. The method of claim 1, wherein the subject is a mammal.

34. The method of claim 1, wherein the subject is a human.

35. The method of claim 5, wherein the miR-155 specific inhibitor comprises LNA-miR-155 specific inhibitor.

36. A pharmaceutical formulation for treating or preventing acute graft-versus-host disease (aGVHD) in a subject, comprising a miR-155 specific inhibitor.

37. The pharmaceutical composition of the claim 36, wherein the pharmaceutical composition is formulated for controlled or sustained release.

38. The method of claim 36, wherein the miR-155 specific inhibitor composition is dispersed in a pharmaceutically acceptable carrier.

39. The method of claim 1, wherein the miR-155 specific inhibitor is human miR-155 specific inhibitor.

40. The method of claim 1, wherein the miR-155 specific inhibitor is recombinant miR-155 specific inhibitor.

41. The method of claim 8, wherein the amount of the miR-155 specific inhibitor composition that is administered is about 1 mg to about 20 g per day.

42. The method of claim 8, wherein the amount of the miR-155 specific inhibitor composition that is administered is about 0.1 g to about 5 g per day.

43. The method of claim 1, further comprising: administering at least one immunosuppressive treatment, wherein the immunosuppressive treatment is selected from: corticosteroids, calcineurin inhibitors, methotrexate, cyclosporine, antithymocyte globulin, prednisolone, methylprednisolone, extracorporeal photopheresis, anti-tumour necrosis factor α antibodies, etanercept, infliximab, mammalian target of rapamycin (mTOR) inhibitors, sirolimus, mycophenolate mofetil, interleukin-2 receptor antibodies, alemtuzumab, pentostatin, and antihistamines.

44. A method of transplanting an allogeneic graft into a human subject in need thereof, comprising:
administering to the subject an effective amount of one or more of:
i) at least one miR-155 specific inhibitor;
ii) active allo-reactive donor human cells which do not express sufficient miR-155 to upregulate T cell expression in the human subject; and,
iii) active auto-reactive human cells which do not express sufficient miR-155 to upregulate T cell expression in the human subject;
and, transplanting the allogeneic graft into the subject.

45. A method of preventing or reducing acute graft-versus-host disease (aGVHD) in a human subject undergoing allogeneic graft, comprising:
administering to the subject an effective amount of one or more of:
i) at least one miR-155 specific inhibitor;
ii) active allo-reactive donor human cells which do not express sufficient miR-155 to upregulate T cell expression in the human subject; and,
iii) active auto-reactive human cells which do not express sufficient miR-155 to upregulate T cell expression in the human subject;
and, transplanting the allogeneic graft into the subject,
wherein the administration of one or more of i), ii) and iii) prevents or reduces aGVHD in the subject.

46. A method of treating or preventing an infection in a subject undergoing allogeneic graft, comprising:
administering to the subject an effective amount of one or more of:
i) at least one miR-155 specific inhibitor;
ii) active allo-reactive donor human cells which do not express sufficient miR-155 to upregulate T cell expression in the human subject; and,
iii) active auto-reactive human cells which do not express sufficient miR-155 to upregulate T cell expression in the human subject;
and, transplanting into the subject the allogeneic graft,
wherein the administration of one or more of i), ii) and iii) prevents or reduces acute graft-versus-host disease (aGVHD) in the subject.

47. A method of increasing engraftment or for reducing graft rejection in a human subject undergoing allogeneic graft, comprising:
administering to the subject an effective amount of one or more of:
i) at least one miR-155 specific inhibitor;
ii) active allo-reactive donor human cells which do not express sufficient miR-155 to upregulate T cell expression in the human subject; and,
iii) active auto-reactive human cells which do not express sufficient miR-155 to upregulate T cell expression in the human subject;
and, transplanting into the human subject the allogeneic graft,
wherein the administration of one or more of i) and ii) prevents or reduces acute graft-versus-host disease (aGVHD) in the subject.

48. The method of claim 44, wherein one or more of: i) the miR-155 specific inhibitor; ii) active allo-reactive donor human cells; and, iii) active auto-reactive human cells; and, the allogeneic graft are administered into the human subject simultaneously.

49. The method of claim 44, wherein one or more of: i) the miR-155 specific inhibitor; ii) active allo-reactive donor human cells; and, iii) active auto-reactive human cells are administered prior to the allogeneic graft.

50. The method of claim 44, wherein the active allo-reactive cells are prepared by: providing cells from an allo-reactive human donor; removing a substantial amount of miR-155 from the cells; and, collecting the miR-155 reduced cells.

51. The method of claim 44, wherein the graft is bone, bone marrow, cornea, heart valve, skin, tendons, stem cells or veins.

52. The method of claim 44, wherein the graft is eye, kidney, heart, lung, liver, intestine, pancreas, spleen or thymus.

53. The method of claim 44, further comprising treating ex vivo the donor organ or tissue with the miR-155 specific inhibitor prior to transplantation in the subject.

54. A method of treating a subject undergoing cancer treatment, the method comprising:
providing immunocompetent donor cells;
inducing graft versus tumor (GvT) response in the subject by transfusing the donor cells to the subject; and
mitigating aGVHD by administering a miR-155 specific inhibitor.

55. The method of claim 54, wherein a cancer being treated is selected from the group consisting of: leukemia, acute myeloid leukemia (AML), chronic lymphocytic leukemia (CLL), chronic myelogenous leukemia (CML), lymphoma, myeloma, and neuroblastoma.

56. The method of claim 54 wherein the subject is immunocompromised at the time of transfusion; and, wherein the donor cells are T cell replete.

57. The method of claim 54 wherein the donor cells comprise, or are derived from, at least one of: T cells, hematopoietic stem cells, bone marrow, spleen, peripheral blood, umbilical cord blood, amniotic fluid, and dental pulp.

58. The method of claim 54 wherein the miR-155 specific inhibitor comprises an 8-mer seed-targeting LNA-antimiR-155 with the sequence: 5′-TAGCATTA-3′.
